SEQUENCE LISTING (63 sequences)

SEQ ID NO: 1 - DNA, 29834 bp, Artificial Sequence
  synthetic construct
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttatattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacctttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgttcttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcagtgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactttttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccattgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacgtagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagatctcatttgcgctcaaaaatttaacggacttacagttttaccacctttacttactgacgaaatgattgcgcaatatacatccgcattgttagccggaactattacatccggatggacttttggcgcaggcgtagcattacagattccattcgctatgcaaatggcttataggtttaacggtataggcgttacgcaaaacgtactttatgagaatcaaaaacttatcgctaaccaatttaattccgctatcggtaagattcaggattcattgtctagtactgctagtgcactcggtaagttgcaagacgtagtgaatcaaaacgctcaagcacttaatacactcgttaaacagcttagttctaattttggcgcaatttctagtgtgcttaacgatatactatctagactcgataaagtcgaagccgaagtgcaaatcgatagattgattaccggtaggttgcaatcattgcaaacatacgttacacagcaattgattagggccgcagagatacgcgctagcgctaatctcgcagctactaaaatgtctgaatgcgtactcggacaatctaaacgtgtcgatttttgcggtaagggatatcatcttatgtcttttccacaatctgcacctcacggagtcgtgtttttacacgttacttatgtgccagctcaagagaaaaattttacaaccgctcctgctatttgtcatgacggtaaggcacattttcctagagagggcgtattcgtttctaacggtacacattggttcgttacacaacgtaatttttacgaacctcaaattattactactgataatacattcgtatcaggtaattgtgacgtagtgataggtatcgttaataatacagtttacgatccacttcaacctgaactcgatagttttaaagaggaactcgataagtattttaaaaatcatacatcacctgacgtcgacttaggcgatatttcaggtattaacgctagtgtcgttaacattcaaaaagagattgatagacttaacgaagtcgctaaaaatcttaacgaatcacttatcgatctgcaagagttaggtaagtatgagcaatatattaaatggccttggtatatttggttaggctttatagccggattgatcgcaatcgttatggttacaattatgttatgttgtatgacatcatgttgttcatgtcttaagggatgttgttcatgcggatcatgttgtaaatttgacgaagacgattccgaaccagtgcttaaaggcgttaagttacattatacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgac

SEQ ID NO: 2 - protein, 12 aa, Artificial Sequence
  synthetic construct
TNSPRRARSVAS

SEQ ID NO: 3 - DNA, 48 bp, Artificial Sequence
  synthetic construct
gataatacgactcactatagattaaaggtttataccttcccaggtaac

SEQ ID NO: 4 - DNA, 23 bp, Artificial Sequence
  synthetic construct
gatgccaaaataatggcgatctc

SEQ ID NO: 5 - DNA, 22 bp, Artificial Sequence
  synthetic construct
gttggttgccataacaagtgtg

SEQ ID NO: 6 - DNA, 27 bp, Artificial Sequence
  synthetic construct
ctaattgaggttgaacctcaacaattg

SEQ ID NO: 7 - DNA, 28 bp, Artificial Sequence
  synthetic construct
gagtatggtactgaagatgattaccaag

SEQ ID NO: 8 - DNA, 24 bp, Artificial Sequence
  synthetic construct
ctaggtggaatgtggtaggattac

SEQ ID NO: 9 - DNA, 29 bp, Artificial Sequence
  synthetic construct
gctgttacagcgtataatggttatcttac

SEQ ID NO: 10 - DNA, 29 bp, Artificial Sequence
  synthetic construct
gctggtttaagtataatgtctcctacaac

SEQ ID NO: 11 - DNA, 28 bp, Artificial Sequence
  synthetic construct
gcacaaaaccagttgaaacatcaaattc

SEQ ID NO: 12 - DNA, 28 bp, Artificial Sequence
  synthetic construct
gcaactagtgttttgagtttttccattg

SEQ ID NO: 13 - DNA, 25 bp, Artificial Sequence
  synthetic construct
gtgaagaatcatctgcaaaatcagc

SEQ ID NO: 14 - DNA, 32 bp, Artificial Sequence
  synthetic construct
caaatgatataagcaattgttatccagaaagg

SEQ ID NO: 15 - DNA, 35 bp, Artificial Sequence
  synthetic construct
gcctttaatactttactattccttatgtcattcac

SEQ ID NO: 16 - DNA, 27 bp, Artificial Sequence
  synthetic construct
ccagacaaactagtatcaaccatatcc

SEQ ID NO: 17 - DNA, 25 bp, Artificial Sequence
  synthetic construct
gctatgggtattattgctatgtctg

SEQ ID NO: 18 - DNA, 21 bp, Artificial Sequence
  synthetic construct
cctacaaggtggttccagttc

SEQ ID NO: 19 - DNA, 19 bp, Artificial Sequence
  synthetic construct
cgacagatgtcttgtgctg

SEQ ID NO: 20 - DNA, 25 bp, Artificial Sequence
  synthetic construct
ggtatccagttgaaactacaaatgg

SEQ ID NO: 21 - DNA, 23 bp, Artificial Sequence
  synthetic construct
gatcagacataccacccaaattg

SEQ ID NO: 22 - DNA, 34 bp, Artificial Sequence
  synthetic construct
cttatgtattgtaagtacaaatgaaagacatcag

SEQ ID NO: 23 - DNA, 27 bp, Artificial Sequence
  synthetic construct
ggtgatgattatgtgtaccttccttac

SEQ ID NO: 24 - DNA, 27 bp, Artificial Sequence
  synthetic construct
ctgttaattgcagatgaaacatcatgc

SEQ ID NO: 25 - DNA, 29 bp, Artificial Sequence
  synthetic construct
gtgtgtagacttatgaaaactataggtcc

SEQ ID NO: 26 - DNA, 21 bp, Artificial Sequence
  synthetic construct
catacaaactgccaccatcac

SEQ ID NO: 27 - DNA, 31 bp, Artificial Sequence
  synthetic construct
ccttgtagtgacaaagcttataaaatagaag

SEQ ID NO: 28 - DNA, 21 bp, Artificial Sequence
  synthetic construct
ctggtgcaactcctttatcag

SEQ ID NO: 29 - DNA, 28 bp, Artificial Sequence
  synthetic construct
gcaaagaatgctattagaaaagtgtgac

SEQ ID NO: 30 - DNA, 32 bp, Artificial Sequence
  synthetic construct
gatagattcctttttctacagtgaaggatttc

SEQ ID NO: 31 - DNA, 23 bp, Artificial Sequence
  synthetic construct
gactcctggtgattcttcttcag

SEQ ID NO: 32 - DNA, 23 bp, Artificial Sequence
  synthetic construct
ctctagcagcaatatcaccaagg

SEQ ID NO: 33 - DNA, 28 bp, Artificial Sequence
  synthetic construct
gcacaagtcaaacaaatttacaaaacac

SEQ ID NO: 34 - DNA, 28 bp, Artificial Sequence
  synthetic construct
caaaaggtgtgagtaaactgttacaaac

SEQ ID NO: 35 - DNA, 19 bp, Artificial Sequence
  synthetic construct
ctcactccctttcggatgg

SEQ ID NO: 36 - DNA, 32 bp, Artificial Sequence
  synthetic construct
gaggtttatgatgtaatcaagattccaaatgg

SEQ ID NO: 37 - DNA, 29 bp, Artificial Sequence
  synthetic construct
gctacaggattggcaactataaattaaac

SEQ ID NO: 38 - DNA, 22 bp, Artificial Sequence
  synthetic construct
ccattctagcaggagaagttcc

SEQ ID NO: 39 - DNA, 21 bp, Artificial Sequence
  synthetic construct
gcaatcctgctaacaatgctg

SEQ ID NO: 40 - DNA, 53 bp, Artificial Sequence
  synthetic construct
tttttttttttttttttttttttttgtcattctcctaagaagctattaaaatc

SEQ ID NO: 41 - DNA, 55 bp, Artificial Sequence
  synthetic construct
gaataatacgactcactatagggacgttcgtgttgttttagatttcatctaaacg

SEQ ID NO: 42 - DNA, 55 bp, Artificial Sequence
  synthetic construct
tttttttttttttttttttttgtcattctcctaagaagctattaaaatcacatgg

SEQ ID NO: 43 - DNA, 26 bp, Artificial Sequence
  synthetic construct
agcttatcatcgataagcttgctagc

SEQ ID NO: 44 - DNA, 24 bp, Artificial Sequence
  synthetic construct
tgtcagtaattccaatgcagtgag

SEQ ID NO: 45 - DNA, 24 bp, Artificial Sequence
  synthetic construct
atgactggaagaatgggtgaaagg

SEQ ID NO: 46 - DNA, 25 bp, Artificial Sequence
  synthetic construct
agaggctttcactattgatgcaagc

SEQ ID NO: 47 - DNA, 24 bp, Artificial Sequence
  synthetic construct
atcaaggatgcgccatcaactttg

SEQ ID NO: 48 - DNA, 24 bp, Artificial Sequence
  synthetic construct
aagtctcacctcagccatagtgac

SEQ ID NO: 49 - DNA, 21 bp, Artificial Sequence
  synthetic construct
aacgccttgtgctgaccctag

SEQ ID NO: 50 - DNA, 25 bp, Artificial Sequence
  synthetic construct
ttggttccaacatcctgtaagttag

SEQ ID NO: 51 - DNA, 20 bp, Artificial Sequence
  synthetic construct
atcttggccgagtgcgcacg

SEQ ID NO: 52 - DNA, 21 bp, Artificial Sequence
  synthetic construct
tcggggatcacaaccaccatc

SEQ ID NO: 53 - DNA, 24 bp, Artificial Sequence
  synthetic construct
tgctgtttatactggctggactac

SEQ ID NO: 54 - DNA, 22 bp, Artificial Sequence
  synthetic construct
tggcatgtatggagctaacacc

SEQ ID NO: 55 - DNA, 25 bp, Artificial Sequence
  synthetic construct
atcatcaccttcaaggacaaaactg

SEQ ID NO: 56 - DNA, 21 bp, Artificial Sequence
  synthetic construct
atccgtgctcagtgagccatg

SEQ ID NO: 57 - DNA, 23 bp, Artificial Sequence
  synthetic construct
agcctacatggatgtcataagtc

SEQ ID NO: 58 - DNA, 26 bp, Artificial Sequence
  synthetic construct
agtggttttgtgtttgtcatccaaag

SEQ ID NO: 59 - DNA, 1794 bp, Artificial Sequence
  synthetic construct
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcggatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 60 - DNA, 1794 bp, Artificial Sequence
  synthetic construct
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatccttgaaaacagctcttactggcgcaatgagggttacaaaggacacaaatgacaacaacctttacaaactacatggtggacatgtttcttgcagagtgaaattgtcagctttgacactcaaggggacatcctacaaaatatgcactgacaaaatgttttttgtcaagaacccaactgacactggccatggcactgttgtgatgcaggtgaaagtgtcaaaaggagccccctgcaggattccagtgatagtagctgatgatcttacagcggcaatcaataaaggcattttggttacagttaaccccatcgcctcaaccaatgatgatgaagtgctgattgaggtgaacccaccttttggagacagctacattatcgttgggagaggagattcacgtctcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcggatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 61 - DNA, 1794 bp, Artificial Sequence
  synthetic construct
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggatttcattgagggggtgcatggaggaacttgggtttcagctaccctggagcaagacaagtgtgtcactgttatggcccctgacaagccttcattggacatctcactagagacagtagccattgatagacctgctgaggtgaggaaagtgtgttacaatgcagttctcactcatgtgaagattaatgacaagtgccccagcactggagaggcccacctagctgaagagaacgaaggggacaatgcgtgcaagcgcacttattctgatagaggctggggcaatggctgtggcctatttgggaaagggagcattgtggcatgcgccaaattcacttgtgccaaatccatgagtttgtttgaggttgatcagaccaaaattcagtatgtcatcagagcacaattgcatgtaggggccaagcaggaaaattggactaccgacattaagactctcaagtttgatgccctgtcaggctcccaggaagtcgagttcattgggtatggaaaagctacactggaatgccaggtgcaaactgcggtggactttggtaacagttacatcgctgagatggaaacagagagctggatagtggacagacagtgggcccaggacttgaccctgccatggcagagtggaagtggcggggtgtggagagagatgcatcatcttgtcgaatttgaacctccgcatgccgccactatcagagtactggccctgggaaaccaggaaggctcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactgacataccaatggcataaagagggatcgtcaatcggtaagttgtttacacagactatgaaaggggtggagagattggccgttatgggcgataccgcttgggactttagttccgccggagggttttttactagcgtcggaaaggggatacataccgtattcggatccgcttttcaggggttgttcggcggactgaattggattacgaaagtgattatgggcgccgtacttatttgggtggggattaacactaggaatatgactatgtctatgtctatgatactagtcggagtgattatgatgtttctgtcattgggcgtaggcgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 62 - DNA, 1794 bp, Artificial Sequence
  synthetic construct
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactgacataccaatggcataaagagggatcgtcaatcggtaagttgtttacacagactatgaaaggggtggagagattggccgttatgggcgataccgcttgggactttagttccgccggagggttttttactagcgtcggaaaggggatacataccgtattcggatccgcttttcaggggttgttcggcggactgaattggattacgaaagtgattatgggcgccgtacttatttgggtggggattaacactaggaatatgactatgtctatgtctatgatactagtcggagtgattatgatgtttctgtcattgggcgtaggcgctgatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

SEQ ID NO: 63 - DNA, 1794 bp, Artificial Sequence
  synthetic construct
atgactggaagaatgggtgaaaggcaactccaaaagattgagagatggttcgtgaggaaccccttttttgcagtgacggctctgaccattgcctaccttgtgggaagcaacatgacgcaacgagtcgtgattgccctactggtcttggctgttggtccggcctactcagctcactgcattggaattactgacagggattttatcgagggggtgcatggcggaacttgggttagcgctacactcgaacaggacaaatgcgttaccgttatggcccccgataagcctagcctagacattagtctcgaaaccgttgcgatcgatagacccgccgaagtgagaaaagtgtgttataacgccgtactgactcacgttaagattaacgacaaatgccctagtacaggcgaagcgcatctagccgaagagaacgagggcgataacgcatgcaaacgtacttatagcgatagggggtgggggaacggatgcggattgttcggtaaggggtcaatcgtcgcatgcgctaagtttacatgcgctaagtctatgtcattgttcgaagtcgatcagactaagattcagtacgtgattagagcgcaattgcatgtgggagcgaaacaggagaattggactactgacattaagacactgaaattcgacgcccttagcggatcacaggaggtcgagtttattgggtacggaaaagcgacactcgagtgtcaggtgcagactgccgttgactttggcaattcatacatagccgaaatggagacagagtcatggatcgttgacagacagtgggcccaggatctgacattgccatggcaatccggatccggaggcgtttggcgcgaaatgcatcatctagtcgagttcgaaccgccacatgccgctacaatcagagtgttggccctaggcaatcaggagggatcccttaaaaccgcattgactggcgctatgcgcgttactaaggacactaacgacaataacctatacaaactgcatggggggcatgtgtcttgtagagtgaaattgtccgcccttacacttaaggggactagctataagatatgcactgacaaaatgtttttcgttaaaaaccctaccgataccggacacggaacagtcgttatgcaggtgaaagtgtcaaaaggcgcaccatgtaggatacccgtaatcgttgccgacgatctgactgccgcaatcaataaggggatactcgtgacagtgaaccctatcgctagcactaacgacgacgaagtgttgatcgaagtgaatccaccttttggcgactcatacattatcgtaggcagaggcgatagtagactcacttaccagtggcacaaagagggaagctcaataggaaagttgttcactcagaccatgaaaggcgtggaacgcctggccgtcatgggagacaccgcctgggatttcagctccgctggagggttcttcacttcggttgggaaaggaattcatacggtgtttggctctgcctttcaggggctatttggcggcttgaactggataacaaaggtcatcatgggggcggtacttatatgggttggcatcaacacaagaaacatgacaatgtccatgagcatgatcttggtaggagtgatcatgatgtttttgtctctaggagttggggcggatcaaggatgcgccatcaactttggcaagagagagctcaagtgcggagatggtatcttcatatttagagactctgatgactggctgaacaagtactcatactatccagaagatcctgtgaagcttgcatcaatagtgaaagcctct

(19) United States
(12) Patent Application Publication
(10) Pub. No.: US 2023/0340423 A1
(43) Pub. Date: Oct. 26, 2023

(54) METHOD OF PRODUCING MODIFIED VIRUS GENOMES AND PRODUCING MODIFIED VIRUSES

(71) Applicant: CODAGENIX INC., Farmingdale, NY (US)

(72) Inventors: John Robert Coleman, Blauvelt, NY (US); Steffen Mueller, Great Neck, NY (US); Chen Yang, Plainview, NY (US); Ying Wang, South Setauket, NY (US); Yutong Song, Stony Brook, MD (US)

(73) Assignee: CODAGENIX INC., Farmingdale, NY (US)

(21) Appl. No.: 18/010,740

(22) PCT Filed: Jul. 7, 2021
(86) PCT No.: PCT/US2021/040716

§ 371 Date: Dec. 15, 2022

Related U.S. Application Data
(60) Provisional application No. 63/048,947, filed on Jul. 7, 2020.

(51) Int. Cl. C12N 7/00; C12Q 1/70; C12Q 1/686

(52) U.S. Cl. CPC C12N 7/00; C12Q 1/70; C12Q 1/686; C12N 2770/20021; C12N 2770/24121

(57) ABSTRACT

The present invention describes methods of generating a modified viral genome, producing infectious RNA, and generating modified viruses. The modified viral genome, infections RNA, and modified viruses comprise deoptimized nucleic acids; for example, codon-pair deoptimized or synonymous codon deoptimized. These modified viruses can be used in vaccines and methods of eliciting a protective immune response.

### CROSS-REFERENCE TO RELATED APPLICATIONS

This application includes a claim of priority under 35 U.S.C. § 119(e) to U.S. provisional patent application No. 63/048,947, filed Jul. 7, 2020, the entirety of which is hereby incorporated by reference.

### FIELD OF INVENTION

This invention relates to producing modified virus genomes such as deoptimized viral genomes, and producing modified viruses such as deoptimized viruses.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Traditional methods employed in virology makes extensive and laborious use of site-directed mutagenesis to make and explore the impact of small sequence variations in the genomes of virus strains, or there is a need to utilize a bacterial or yeast host organism. As such, there is a need in the art for methods of synthesizing and recovery of synthetic viruses, for example, SARS-CoV-2 viruses and Yellow Fever Viruses, among others, wherein region(s) of the wild type virus are replaced with modified sequences, and to sidestep genetic instability and toxicity problem that have plagued traditional cloning methods in the past.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of generating a modified viral genome, comprising performing reverse transcription polymerase chain reaction (“RT-PCR”) on a viral RNA from an RNA virus to generate cDNA; performing polymerase chain reaction (“PCR”) to generate and amplify two or more overlapping cDNA fragments from the cDNA, wherein the two or more overlapping cDNA fragments collectively encode the RNA virus; substituting one or more overlapping cDNA fragments comprising a modified sequence for one or more corresponding overlapping cDNA fragment generated from the viral RNA; and performing overlapping and amplifying PCR to construct the modified viral genome, wherein the modified viral genome comprises one or more modified sequences.

Various embodiments provide for a method of generating a modified viral genome, comprising performing polymerase chain reaction (“PCR”) to generate and amplify two or more overlapping cDNA fragments from cDNA encoding viral RNA from an RNA virus, wherein the two or more overlapping cDNA fragments collectively encode the RNA virus, wherein one or more overlapping cDNA fragments comprises a modified sequence; and performing overlapping and amplifying PCR to construct the modified viral genome, wherein the modified viral genome comprises one or more modified sequences.

In various embodiments, these methods can further comprise extracting the viral RNA from the RNA virus prior to performing RT-PCR.

In various embodiments of these methods, each of the one or more overlapping cDNA fragments comprising the modified sequence can comprise (1) a recoded sequence having reduced codon pair bias compared to a corresponding sequence on the cDNA, (2) an increased number of CpG or UpA di-nucleotides compared to a corresponding sequence on the cDNA; or (3) at least 5 codons substituted with synonymous codons less frequently used.

In various embodiments of these methods, performing PCR to generate and amplify two or more overlapping cDNA fragments from the cDNA can comprise using two or more primer pairs selected from Table 1. In various embodiments of these methods, performing PCR to generate and amplify 10 or more overlapping cDNA fragments from the cDNA can comprise using 10 or more primer pairs selected from Table 1. In various embodiments of these methods, performing PCR to generate and amplify 15 or more overlapping cDNA fragments from the cDNA can comprise using 15 or more primer pairs selected from Table 1. In various embodiments of these methods, performing PCR to generate and amplify 19 overlapping cDNA fragments from the first cDNA can comprise using all 19 primer pairs from Table 1.

In various embodiments of these methods, performing PCR to generate and amplify two or more overlapping cDNA fragments from the cDNA can comprise using two or more primer pairs selected from Table 2. In various embodiments of these methods, performing PCR to generate and amplify 5 or more overlapping cDNA fragments from the cDNA can comprise using 5 or more primer pairs selected from Table 2. In various embodiments of these methods, performing PCR to generate and amplify 8 or more overlapping cDNA fragments from the cDNA can comprise using 8 or more primer pairs selected from Table 2.

In various embodiments of these methods, the two or more overlapping cDNA fragments from the cDNA can be 5 or more overlapping cDNA fragments and the 5 or more overlapping cDNA fragments collectively encode the RNA virus. In various embodiments of these methods, the two or more overlapping cDNA fragments from the cDNA can be 8 or more overlapping cDNA fragments and the 8 or more overlapping cDNA fragments collectively encode the RNA virus. In various embodiments of these methods, the two or more overlapping cDNA fragments from the cDNA can be 10 or more overlapping cDNA fragments and the 10 or more overlapping cDNA fragments collectively encode the RNA virus. In various embodiments of these methods, the two or more overlapping cDNA fragments from the cDNA can be 15 or more overlapping cDNA fragments and the 15 or more overlapping cDNA fragments collectively encode the RNA virus. In various embodiments of these methods, the two or more overlapping cDNA fragments from the cDNA can be 19 overlapping cDNA fragments and the 19 overlapping cDNA fragments collectively encode the RNA virus.

In various embodiments of these methods, the viral RNA can be from a wild-type RNA virus, and the cDNA is cDNA encoding the viral RNA from the wild-type RNA virus (“wild-type cDNA”).

In various embodiments of these methods, the viral RNA can be from SARS-CoV-2, SARS-CoV-2 variant, or Yellow Fever virus.

In various embodiments of these methods, each of the primers can be about 15-65 base pairs (bp) in length. In various embodiments of these methods, each of the primers can be about 15-55 base pairs (bp) in length.

In various embodiments of these methods, each overlap between the two or more overlapping cDNA fragments can overlap by about 40-400 bp. In various embodiments of these methods, n each overlap between the two or more overlapping cDNA fragments can overlap by about 100-300 bp.

In various embodiments of these methods, the methods can comprise performing RT-PCR on viral RNA from a wild-type RNA virus to generate cDNA (“wild-type cDNA”); performing PCR to generate and amplify 19 overlapping cDNA fragments from the wild-type cDNA, wherein the 19 overlapping cDNA fragments collectively encode the wild-type RNA virus; substituting an overlapping cDNA fragment comprising a deoptimized sequence for a corresponding overlapping cDNA fragment from the wild-type cDNA; and performing overlapping and amplifying PCR to construct the modified viral genome comprising the deoptimized sequence.

Various embodiments of the present invention provide for a method of generating a modified infectious RNA, comprising: performing in vitro transcription of a modified viral genome to generate a modified RNA transcript.

In various embodiments, these methods can further comprise performing any one of the methods described herein to generate the modified viral genome before performing the in vitro transcription.

Various embodiments of the present invention provide for a method of generating a modified virus, comprising transfecting host cells with a quantity of a modified infectious RNA; culturing the host cells; and collecting infection medium comprising the modified virus.

In various embodiments, these methods can further comprise performing any one of the methods of the present invention as described herein to obtain the quantity of modified infectious RNA before transfecting host cells with the quantity of the modified infectious RNA.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts a schematic of recovery of deoptimized SARS-CoV-2 construct (CDX-005).

FIG. 2A depicts purified genome fragments 1-19 generated from viral cDNA compared to a 1 kB Plus ladder (NEB). Fragments 1-18 (1.8 kb) and 19 (1.2 kb) were the expected sizes.

FIG. 2B depicts re-constructed WW-WWW and WW-WWD full-length genomic DNA generated by overlapping PCR next to lambda DNA digested with Afl II (Top band, 30 Kb) was also the expected size.

FIG. 3 depicts plaque phenotype of wildtype (left) and CDX-005 (right) strains of SARS-CoV-2 on Vero E6 cells. CDX-005 produces smaller plaques and grows to 40% lower titers on Vero E6 cells as compared to wildtype virus.

FIG. 4 depicts various representative versions of the codon-pair deoptimized (CPD) Yellow Fever 17D Viral Genome design.

FIG. 5 depicts PCR gel check for F1-F8 for the building the deoptimized YFV. F2 can be either of the wild-type (Wt) or any one of CPD-fragments (DW, WD, DD, or DDDW).

FIG. 6 depicts gel check for four full length CPD YF genome PCR (˜11 kb).

FIG. 7 depicts RNA gel check for four full length YF-CPD genome RNAs.

FIG. 8 plaque assay for the vaccine strain YF-17D (left column) and the recovered YF-DW viral variant (right column) at 33° C. (top row) and 37° C. (bottom row).

FIG. 9 depicts plaque assay for the vaccine strain YF-(left column) and the recovered YF-DDDW viral variant (right column) at 33° C. (top row) and 37° C. (bottom row).

FIGS. 10A-10D depict detection of Infected Vero Cells by Immunohistochemical Staining. Cells transfected with (A) YF-DD RNA or (B) no RNA were fixed with Methanol/Acetone 8 days after RNA transfection. Cells infected with (C) day 4 YF-DD transfection supernatants or (D) mock supernatant were fixed with Methanol/Acetone 8 days after infection. YF-infected cells were visualized by IHC staining with mouse mAb anti-Flavivirus Group Antigen, clone D1-4G2-4-15 (ATCC® HB-112), in conjunction with HRP-labeled goat anti-mouse secondary antibody and VECTOR VIP chromogenic substrate.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Parent virus” as used herein refer to a reference virus to which a recoded nucleotide sequence is compared for encoding the same or similar amino acid sequence.

“SARS-CoV-2” refers to a coronavirus that has a wild-type sequence, natural isolate sequence, or mutant forms of the wild-type sequence or natural isolate sequence that causes COVID-19. Mutant forms arise naturally through the virus' replication cycles, or through genetic engineering.

“SARS-CoV-2 variant” as used herein refers to a mutant form of SARS-CoV-2 that has developed naturally through the virus' replication cycles as it replicates in and/or transmits between hosts such as humans. Examples of SARS-CoV-2 variants include but are not limited to Alpha variant (also known as U.K. variant, 20I/501Y.V1, VOC 202012/01, or B.1.1.7), Beta variant (also known as South African variant, 20H/501Y.V2, or B.1.351), Delta variant (B.1.617.2), and Gamma variant (also known as Brazil variant or P.1).

“Natural isolate” as used herein with reference to SARS-CoV-2 refers to a virus such as SARS-CoV-2 that has been isolated from a host (e.g., human, bat, feline, pig, or any other host) or natural reservoir. The sequence of the natural isolate can be identical or have mutations that arose naturally through the virus' replication cycles as it replicates in and/or transmits between hosts, for example, humans.

“Washington coronavirus isolate” as used herein refers to a wild-type isolate of SARS-CoV-2 that has GenBank accession no. MN985325.1 as of Jul. 5, 2020, which is herein incorporated by reference as though fully set forth in its entirety.

“Frequently used codons” or “codon usage bias” as used herein refer to differences in the frequency of occurrence of synonymous codons in coding DNA for a particular species, for example, human, a particular virus, coronavirus, SARS-CoV-2, or Yellow Fever Virus.

“Codon pair bias” as used herein refers to synonymous codon pairs that are used more or less frequently than statistically predicted in a particular species, for example, human, a particular virus, coronavirus, SARS-CoV-2, or Yellow Fever Virus.

A “subject” as used herein means any animal or artificially modified animal Animals include, but are not limited to, humans, non-human primates, cows, horses, sheep, pigs, dogs, cats, rabbits, ferrets, rodents such as mice, rats and guinea pigs, bats, snakes, and birds. Artificially modified animals include, but are not limited to, SCID mice with human immune systems. In a preferred embodiment, the subject is a human.

A “viral host” means any animal or artificially modified animal, or insect that a virus can infect. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems. In a specific embodiment, the viral host is a human. Embodiments of birds are domesticated poultry species, including, but not limited to, chickens, turkeys, ducks, and geese. Insects include, but are not limited to mosquitos.

Described herein, we generated wildtype SARS-CoV-2 and variant SARS-CoV-2 from genome segments rescued from extracted viral RNA and were successful in incorporating a synthetic fragment into the rescued viral cDNA to derive a partially synthetic vaccine candidate S-WWD. Herein we show our overlapping PCR based synthesis approach and transfection protocols under BSL-3 conditions for betacoronaviruses. We have generated a potential vaccine candidate and have confirmed the success of our experimental protocols. Additionally, we have in vitro evidence of S-WWD attenuation based on reduced plaque size and virus yield. Also described herein, we generated Yellow Fever from genome segments and were successful in incorporating various versions of synthetic/deoptimized fragments into the rescued viral cDNA to derive several Yellow Fever vaccines.

With respect to SARS-CoV-2, and for the sake of speed in view of the ongoing SARS-CoV-2 pandemic we used a cDNA derived from a clinical isolate (USA-WA1/2020) as the donor of most of the genetic elements for our reassortant viruses. We divided the genome into 19 approximately 1.8 kb fragments, each fragment overlapping with their respective neighbors by about 200 bp. The fragments size of 1,800 bp was chosen, as this is currently the common size limit for uncloned de-novo synthesized DNA fragment commercially available (Twist Biosciences). This fragment size therefore allows to mix and match naturally derived viral cDNA fragments with custom designed synthetic DNA blocks without ever needing to clone any recombinant DNA molecule.

We first re-derived the wild type USA-WA1/2020 virus from 19 overlapping viral cDNA fragments that were re-assembled into a full length cDNA genome by overlap PCR, followed by in vitro transcription, and RNA electroporation into Vero E6 cells. The resultant virus CDX-006 was indistinguishable for the natural isolate USA-WA1/2020 in its growth properties and plaque phenotype.

In order to show the utility of this method to create custom genetically modified SARS-CoV-2 viruses, and live attenuated vaccine candidates in particular, we PCR-assembled two SARS-CoV-2 genomes in which one of the 19 viral cDNA-derived fragments (Fragment 14 or 16) were substituted with a corresponding de novo synthesized, SAVE-deoptimized fragment encompassing a portion of the Spike protein encoding sequence, and derived synthetic vaccine candidates CDX-005 and CDX-007. This experiment established proof-of-concept for our overlapping PCR based synthesis approach and transfection protocols under BSL-3 conditions for betacoronaviruses.

We further applied these approaches to SARS-CoV-2 variants and other viruses such as Yellow Fever Virus. These approaches described herein can be applied to other RNA viruses as well.

The methods described herein generates full cDNA that can be used for down-stream viral production. For example, infectious RNA is generated and used to infect/transfect the cells directly to produce the viruses. Further, the methods described herein eliminates the need for intermediate DNA clones such as a plasmid, BAC, YAC or the like. These methods described herein also eliminates the need for a cloning host. The methods are performed in a “test tube” until RNA is transfected into the virus target cells.

With the traditional cloning methods those large DNA constructs are often extremely unstable (genetically) in said cloning hosts (as is the case for CoVs and flavivirus genomes). Due to the sequences often encoding something that is toxic for the cloning host, the host does not tolerate the offending sequences. Generations of researchers have tried to find ways of overcoming this instability. For example, Li et al. J Virol. 2018 Aug. 16; 92(17) uses standard DNA cloning (e.g., plasmid), which is a lengthy and tedious process, including utilizing intermediate DNA clones. Ultimately, their final full length clone is still not stable and a method to overcome it was to introduce an artificial intron in their DNA close to disrupt the offending sequence locus.

Cloning SARS-CoV-2 and flavivirus genomes in those cloning hosts is extremely tedious, wrought with problems, and ultimately often fails. The methods described herein overcome these problems of the traditional methods. The way the inventors recovered the SARS-CoV-2 viruses described herein is unique and remarkable for such a large virus.

As such, various embodiments of the present invention are based, at least in part, on these finding and those further described herein.

Various embodiments of the present invention provide for a method of generating a modified viral genome, comprising performing reverse transcription polymerase chain reaction (“RT-PCR”) on a viral RNA from an RNA virus to generate cDNA; performing polymerase chain reaction (“PCR”) to generate and amplify two or more overlapping cDNA fragments from the cDNA, wherein the two or more overlapping cDNA fragments collectively encode the RNA virus; substituting one or more overlapping cDNA fragments comprising a modified sequence for one or more corresponding overlapping cDNA fragment generated from the viral RNA; and performing overlapping and amplifying PCR to construct the modified viral genome, wherein the modified viral genome comprises one or more modified sequences. In various embodiments, the method comprises performing at least 1 passage of a RNA viral isolate on permissive cells before performing the RT-PCR on the viral RNA from the RNA virus to generate the cDNA.

Various embodiments of the invention provide for a method of generating a modified viral genome, comprising performing polymerase chain reaction (“PCR”) to generate and amplify two or more overlapping cDNA fragments from cDNA encoding viral RNA from an RNA virus, wherein the two or more overlapping cDNA fragments collectively encode the RNA virus; substituting one or more overlapping cDNA fragments comprising a modified sequence for one or more corresponding overlapping cDNA fragment; performing overlapping and amplifying PCR to construct the modified viral genome, wherein the modified viral genome comprises one or more modified sequences.

Various embodiments of the invention provide for a method of generating a modified viral genome, comprising performing polymerase chain reaction (“PCR”) to generate and amplify two or more overlapping cDNA fragments from cDNA encoding viral RNA from an RNA virus, wherein the two or more overlapping cDNA fragments collectively encode the RNA virus, and wherein one or more overlapping cDNA fragments comprises a modified sequence; performing overlapping and amplifying PCR to construct the modified viral genome, wherein the modified viral genome comprises one or more modified sequences.

In various embodiments, the method further comprising extracting the viral RNA from the RNA virus prior to performing RT-PCR. Thus, the method comprises extracting a viral RNA from a RNA virus; performing reverse transcription polymerase chain reaction (“RT-PCR”) on the viral RNA from the RNA virus to generate cDNA; performing polymerase chain reaction (“PCR”) to generate and amplify two or more overlapping cDNA fragments from the cDNA, wherein the two or more overlapping cDNA fragments collectively encode the RNA virus; substituting one or more overlapping cDNA fragments comprising a modified sequence for one or more corresponding overlapping cDNA fragment generated from the viral RNA; and performing overlapping and amplifying PCR to construct the modified viral genome, wherein the modified viral genome comprises one or more modified sequences.

In various embodiments, performing overlapping PCR to construct the modified viral genome is done on the two or more overlapping cDNA fragments at the same time. Thus, if there are 5 more overlapping cDNA fragments, overlapping PCR to construct the modified viral genome is done on those 5 fragments at the same time. As further examples, if there are 8 more overlapping cDNA fragments, overlapping PCR to construct the modified viral genome is done on those 8 fragments at the same time; if there are 10 more overlapping cDNA fragments, overlapping PCR to construct the modified viral genome is done on those 10 fragments at the same time; if there are 15 more overlapping cDNA fragments, overlapping PCR to construct the modified viral genome is done on those 15 fragments at the same time; if there are 19 more overlapping cDNA fragments, overlapping PCR to construct the modified viral genome is done on those 19 fragments at the same time; if there are 20 more overlapping cDNA fragments, overlapping PCR to construct the modified viral genome is done on those 20 fragments at the same time; if there are 25 more overlapping cDNA fragments, overlapping PCR to construct the modified viral genome is done on those 25 fragments at the same time; and if there are 30 more overlapping cDNA fragments, overlapping PCR to construct the modified viral genome is done on those 30 fragments at the same time.

In various embodiments, the RNA virus is a negative strand RNA virus. Examples of negative strand RNA include but are not limited to those of the following families Bornaviridae, Filoviridae, Mymonaviridae, Nyamiviridae, Paramyxoviridae, Pneumoviridae, Rhabdoviridae, Sunviridae, Feraviridae, Fimoviridae, Hantaviridae, Jonviridae, Nairoviridae, Peribunyaviridae, Phasmaviridae, Phenuiviridae, Tospoviridae, Arenaviridae, and Ophioviridae Examples of negative strand RNA viruses include but are not limited to Borna disease virus, Ebola virus, Marburg virus, measles virus, mumps virus, Nipah virus, Hendra virus, respiratory syncytial virus (RSV), metapneumovirus, influenza virus, rabies virus, and Lassa virus. In particular embodiments, the RNA virus is RSV. In other particular embodiments, the RNA virus is influenza virus.

In other embodiments, the RNA virus is a positive strand RNA virus. Example of positive strand RNA include but are not limited to those of following families Abyssoviridae, Arteriviridae, Cremegaviridae, Gresnaviridae, Olifoviridae, Coronaviridae, Medioniviridae, Mesoniviridae, Mononiviridae, Nanghoshaviridae, Nanhypoviridae, Euroniviridae, Roniviridae, Tobaniviridae, Caliciviridae, Dicistroviridae, Iflaviridae, Marnaviridae, Picornaviridae, Polycipiviridae, Secoviridae, Solinviviridae, Alphatetraviridae, Alvernaviridae, Astroviridae, Barnavirida, Benyviridae, Bromoviridae, Caliciviridae, Carmotetraviridae, Closteroviridae, Flaviviridae, Hepeviridae, Leviviridae, Luteoviridae, Narnaviridae, Nodaviridae, Permutotetraviridae, Potyviridae, Sarthroviridae, Solemoviridae, Solinviviridae, Togaviridae, Tombusviridae, Virgaviridae; and the following genera Albetovirus, Aumaivirus, Blunervirus, Cilevirus, Higrevirus, Idaeovirus, Ourmiavirus, Papanivirus, Polemovirus, Sinaivirus, and Virtovirus. Particular examples of positive strand RNA viruses include but are not limited coronavirus, including but not limited to Human coronavirus OC43, Human coronavirus HKU1, Middle East respiratory syndrome-related coronavirus (MERS-CoV), Severe acute respiratory syndrome coronavirus (SARS-CoV), and Severe acute respiratory syndrome coronavirus 2 (SARS-CoV-2) (including its variants). In various embodiments, the SARS-CoV-2 is the Alpha, Beta, Delta, or Gamma variant. Additional examples of positive strand RNA viruses include but are not limited to poliovirus, rhinovirus, hepatitis A virus, norovirus, Yellow fever virus, West Nile Virus, Hepatitis C virus, Dengue fever virus, Zika virus, and Rubella virus. In particular embodiments, the RNA virus is a Yellow fever virus. In yet particular embodiments, the RNA virus is 17D Yellow fever virus. In still other particular embodiments, the RNA virus is 17D-204, 17DD, or 17D-213.

In still other embodiments, the RNA virus is a double-stranded RNA virus. Examples of dsRNA viruses include but are not limited to those of the following families Amalgaviridae, Birnaviridae, Chrysoviridae, Cystoviridae, Endornaviridae, Hypoviridae, Megabirnaviridae, Partitiviridae, Picobirnaviridae, Quadriviridae, Reoviridae, and Totiviridae. An example of dsRNA viruses includes but is not limited to Rotavirus.

In various embodiments, the virus is not Zika virus. In various embodiments, the virus is not Japanese encephalitis virus. In various embodiments, the virus is not West Nile virus. In various embodiments, the virus does not belong to the Flaviviridae family.

In various embodiments, each of the one or more overlapping cDNA fragments comprising the modified sequence comprises (1) a recoded sequence having reduced codon pair bias compared to a corresponding sequence on the cDNA, (2) at least 5 codons substituted with synonymous codons less frequently used, or (3) an increased number of CpG or UpA di-nucleotides compared to a corresponding sequence on the cDNA.

In embodiments wherein the modified sequence comprises a recoded sequence having reduced codon pair bias compared to a corresponding sequence on the cDNA, the recoded sequence has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the codon pair bias of the recoded sequence is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the corresponding sequence on the cDNA. In certain embodiments, it is in comparison corresponding sequence from which the calculation is to be made; for example, the corresponding sequence of a wild type virus.

“Corresponding sequence” as used herein refers to a comparison sequence by which the modified sequence is encoding the same or similar amino acid sequence of the comparison sequence. In various embodiments, the corresponding sequence is a sequence that encodes a viral protein. In various embodiments, the corresponding sequence is at least 50 codons in length. In various embodiments, the corresponding sequence is at least 100 codons in length. In various embodiments, the corresponding sequence is at least 150 codons in length. In various embodiments, the corresponding sequence is at least 200 codons in length. In various embodiments, the corresponding sequence is at least 250 codons in length. In various embodiments, the corresponding sequence is at least 300 codons in length. In various embodiments, the corresponding sequence is at least 350 codons in length. In various embodiments, the corresponding sequence is at least 400 codons in length. In various embodiments, the corresponding sequence is at least 450 codons in length. In various embodiments, the corresponding sequence is at least 500 codons in length. In various embodiments, the corresponding sequence is the viral protein sequence. In various embodiments, the corresponding sequence is the sequence of the entire virus.

In various embodiments, “similar amino acid sequence” as used herein refers to an amino acid sequence having less than 2% amino acid substitutions, deletions or additions compared to the comparison sequence. In various embodiments, if specifically provided for in the claims, “similar amino acid sequence” refers to an amino acid sequence having less than 1.75% amino acid substitutions, deletions or additions compared to the comparison sequence. In various embodiments, if specifically provided for in the claims, “similar amino acid sequence” refers to an amino acid sequence having less than 1.5% amino acid substitutions, deletions or additions compared to the comparison sequence. In various embodiments, if specifically provided for in the claims, “similar amino acid sequence” refers to an amino acid sequence having less than 1.25% amino acid substitutions, deletions or additions compared to the comparison sequence. In various embodiments, if specifically provided for in the claims, “similar amino acid sequence” refers to an amino acid sequence having less than 1% amino acid substitutions, deletions or additions compared to the comparison sequence. In various embodiments, if specifically provided for in the claims, “similar amino acid sequence” refers to an amino acid sequence having less than 0.75% amino acid substitutions, deletions or additions compared to the comparison sequence. In various embodiments, if specifically provided for in the claims, “similar amino acid sequence” refers to an amino acid sequence having less than 0.5% amino acid substitutions, deletions or additions compared to the comparison sequence. In various embodiments, if specifically provided for in the claims, “similar amino acid sequence” refers to an amino acid sequence having less than 0.25% amino acid substitutions, deletions or additions compared to the comparison sequence.

In various embodiments, an amino acid sequence having a deletion of a furin cleavage site in considered a similar amino acid sequence. For example, for SARS-CoV-2, a 36 nt deletion is in the Spike gene (genome position 23594-23629). The deletion encompasses the 12 amino acids TNSPRRARSVAS (SEQ ID NO:2) that include the polybasic furin cleavage site. The furin cleavage site in SARS-CoV2 Spike has been proposed as a potential driver of the highly pathogenic phenotype of SARS-CoV2 in the human host. While not wishing to be bound by any particular theory, we believe that absence of the furin cleavage is beneficial to the SARS-CoV-2 virus growth in vitro in Vero cells, and that the deletion evolved during passaging in Vero cell culture. We further believe that the absence of the furin cleavage site may contribute to attenuation in the human host of a SARS-CoV-2 virus carrying such mutation.

In embodiments wherein the modified sequence comprises at least 5 codons substituted with synonymous codons less frequently used, the modified sequence comprises at least 10, or at least 30, or at least 30, or at least 40, or at least 50, or at least 75, or at least 100, at least 150, or at least 200, or at least 250 substituted with synonymous codons less frequently used. In certain embodiments, the modified sequence comprises at least 20 codons substituted with synonymous codons less frequently used. In certain embodiments, the modified sequence comprises at least 50 codons substituted with synonymous codons less frequently used.

In some embodiments, the substitution of synonymous codons is with those that are less frequent in the viral host; for example, human. Other examples of viral hosts include but are not limited to those noted above. In some embodiments, the substitution of synonymous codons is with those that are less frequent in the virus itself.

In embodiments wherein the modified sequence comprises an increased number of CpG or UpA di-nucleotides compared to a corresponding sequence (for example, on the cDNA), the increase is of about 15-55 CpG or UpA di-nucleotides compared the corresponding sequence. In various embodiments, increase is of about 15, 20, 25, 30, 35, 40, 45, or 55 CpG or UpA di-nucleotides compared the corresponding sequence. In some embodiments, the increased number of CpG or UpA di-nucleotides compared to a corresponding sequence (e.g., on the cDNA) is about 10-75, 15-25, 25-50, or 50-75 CpG or UpA di-nucleotides compared the corresponding sequence.

In various embodiments, performing PCR to generate and amplify two or more overlapping cDNA fragments from the cDNA comprises using two or more primer pairs, each pair specific for each of the overlapping cDNA fragments. In various embodiments, performing PCR to generate and amplify two or more overlapping cDNA fragments from the cDNA comprises using two or more primer pairs selected from Table 1. In various embodiments, performing PCR to generate and amplify two or more overlapping cDNA fragments from the cDNA comprises using two or more primer pairs selected from Table 2.

In various embodiments, the length of the primers is about 15-55 base pairs (bp) in length. In various embodiments, the length of the primers is about 19-55 bp in length. In various embodiments, the length of the primers is about 10-65 bp in length. In various embodiments, the length of the primers is about 16-20, 21-25, 26-30, 31-35, 36-40, 41-45, 46-50, 51-55, 56-60, or 61-65 bp in length.

In various embodiments, performing PCR to generate and amplify two or more overlapping cDNA fragments from the cDNA comprises using 5 or more primer pairs, each pair specific for each of the overlapping cDNA fragments. In various embodiments, the two or more overlapping cDNA fragments from the cDNA is 5 or more overlapping cDNA fragments and the 5 or more overlapping cDNA fragments collectively encode the RNA virus. In various embodiments, performing PCR to generate and amplify 5 or more overlapping cDNA fragments from the cDNA comprises using 5 or more primer pairs selected from Table 1. In various embodiments, performing PCR to generate and amplify 5 or more overlapping cDNA fragments from the cDNA comprises using 5 or more primer pairs selected from Table 2.

In various embodiments, performing PCR to generate and amplify two or more overlapping cDNA fragments from the cDNA comprises using 8 or more primer pairs, each pair specific for each of the overlapping cDNA fragments. In various embodiments, the two or more overlapping cDNA fragments from the cDNA is 8 or more overlapping cDNA fragments and the 8 or more overlapping cDNA fragments collectively encode the RNA virus. In various embodiments, performing PCR to generate and amplify 8 or more overlapping cDNA fragments from the cDNA comprises using 8 or more primer pairs selected from Table 1. In various embodiments, performing PCR to generate and amplify 8 or more overlapping cDNA fragments from the cDNA comprises using 8 or more primer pairs selected from Table 2.

In various embodiments, performing PCR to generate and amplify two or more overlapping cDNA fragments from the cDNA comprises using 10 or more primer pairs, each pair specific for each of the overlapping cDNA fragments. In various embodiments, the two or more overlapping cDNA fragments from the cDNA is 10 or more overlapping cDNA fragments and the 10 or more overlapping cDNA fragments collectively encode the RNA virus. In various embodiments, performing PCR to generate and amplify 10 or more overlapping cDNA fragments from the cDNA comprises using 10 or more primer pairs selected from Table 1.

In various embodiments, performing PCR to generate and amplify two or more overlapping cDNA fragments from the cDNA comprises using 15 or more primer pairs, each pair specific for each of the overlapping cDNA fragments. In various embodiments, the two or more overlapping cDNA fragments from the cDNA is 15 or more overlapping cDNA fragments and the 15 or more overlapping cDNA fragments collectively encode the RNA virus. In various embodiments, performing PCR to generate and amplify 15 or more overlapping cDNA fragments from the cDNA comprises using 15 or more primer pairs selected from Table 1.

In various embodiments, the two or more overlapping cDNA fragments from the cDNA is 20 or more overlapping cDNA fragments and the 20 or more overlapping cDNA fragments collectively encode the RNA virus. In various embodiments, performing PCR to generate and amplify 20 or more overlapping cDNA fragments from the cDNA comprises using 20 or more primer pairs, each pair specific for each overlapping cDNA fragments.

In various embodiments, the two or more overlapping cDNA fragments from the cDNA is 25 or more overlapping cDNA fragments and the 25 or more overlapping cDNA fragments collectively encode the RNA virus. In various embodiments, performing PCR to generate and amplify 25 or more overlapping cDNA fragments from the cDNA comprises using 25 or more primer pairs, each pair specific for each overlapping cDNA fragments.

In various embodiments, the two or more overlapping cDNA fragments from the cDNA is 30 or more overlapping cDNA fragments and the 30 or more overlapping cDNA fragments collectively encode the RNA virus. In various embodiments, performing PCR to generate and amplify 30 or more overlapping cDNA fragments from the cDNA comprises using 30 or more primer pairs, each pair specific for each overlapping cDNA fragments.

In various embodiments, the two or more overlapping cDNA fragments from the cDNA is 19 overlapping cDNA fragments and the 19 overlapping cDNA fragments collectively encode the RNA virus; for example, the SARS-CoV-2 or SARS-CoV-2 variant (e.g., Alpha, Beta, Delta, or Gamma). In various embodiments, performing PCR to generate and amplify 19 overlapping cDNA fragments from the first cDNA comprises using all 19 primer pairs from Table 1.

In various embodiments, the two or more overlapping cDNA fragments from the cDNA is 8 overlapping cDNA fragments and the 8 overlapping cDNA fragments collectively encode the RNA virus, for example, the Yellow Fever Virus (e.g., 17D, 17DD, 17D-213, 17D-204). In various embodiments, performing PCR to generate and amplify 8 overlapping cDNA fragments from the first cDNA comprises using all 8 primer pairs from Table 2.

In various embodiments, the two or more overlapping cDNA fragments is 2-30 fragments. In various embodiments, the two or more overlapping cDNA fragments is 2-5 fragments. In various embodiments, the two or more overlapping cDNA fragments is 6-8 fragments. In various embodiments, the two or more overlapping cDNA fragments is 8-10 fragments. In various embodiments, the two or more overlapping cDNA fragments is 11-15 fragments. In various embodiments, the two or more overlapping cDNA fragments is 16-20 fragments. In various embodiments, the two or more overlapping cDNA fragments is 21-25 fragments. In various embodiments, the two or more overlapping cDNA fragments is 26-30 fragments.

In various embodiments, the length of the overlap is about 40-400 bp. In various embodiments, the length of the overlap is about 200 bp. In various embodiments, the length of the overlap is about 40-100 bp. In various embodiments, the length of the overlap is about 100-200 bp. In various embodiments, the length of the overlap is about 100-150 bp. In various embodiments, the length of the overlap is about 150-200 bp. In various embodiments, the length of the overlap is about 200-250 bp. In various embodiments, the length of the overlap is about 200-300 bp. In various embodiments, the length of the overlap is about 300-400 bp.

In various embodiments, the viral RNA is from a wild-type RNA virus, and the cDNA is cDNA encoding the viral RNA from the wild-type RNA virus (“wild-type cDNA”).

In various embodiments, the viral RNA is from a wild-type SARS-CoV-2, and the cDNA is cDNA encoding the viral RNA from the wild-type SARS-CoV-2. In various embodiments, the viral RNA is from a variant SARS-CoV-2, and the cDNA is cDNA encoding the viral RNA from the variant SARS-CoV-2. In various embodiments, the variant is the Alpha variant, Beta variant, Delta variant, or Gamma variant.

Examples of the Alpha (U.K.) variant include but are not limited to GenBank Accession Nos. MW462650 (SARS-CoV-2/human/USA/MN-MDH-2252/2020), MW463056 (SARS-CoV-2/human/USA/FL-BPHL-2270/2020), and MW440433 (SARS-CoV-2/human/USA/NY-Wadsworth-291673-01/2020), all as of Jan. 19, 2021, all incorporated herein by reference as though fully set forth in their entirety. Additional examples of the U.K. variant include but are not limited to GISAID ID Nos. EPI_ISL_778842 (hCoV-19/USA/TX-CDC-9KXP-8438/2020; 2020-12-28), EPI_ISL_802609 (hCoV-19/USA/CA-CDC-STM-050/2020; 2020-12-28), EPI_ISL_802647 (hCoV-19/USA/FL-CDC-STM-043/2020; 2020-12-26), EPI_ISL_832014 (hCoV-19/USA/UT-UPHL-2101178518/2020; 2020-12-31), EPI_ISL_850618 (hCoV-19/USA/IN-CDC-STM-183/2020; 2020-12-31), and EPI_ISL_850960 (hCoV-19/USA/FL-CDC-STM-A100002/2021; 2021-01-04), all as of Jan. 20, 2021; and EPI_ISL_581117, EPI_ISL_596982, EPI_ISL_599956, EPI_ISL_600093, EPI_ISL_606375, EPI_ISL_606415, EPI_ISL_606424, EPI_ISL_608363, and EPI_ISL_608430, all as of Jun. 28, 2021; and all incorporated herein by reference as though fully set forth in their entirety.

Examples of the Beta (South Africa) variant include but are not limited to GISAID ID Nos. EPI_ISL_766709 (hCoV-19/Sweden/20-13194/2020; 2020-12-24), EPUSL_768828 (hCoV-19/France/PAC-NRC2933/2020; 2020-12-22), EPUSL_770441 (hCoV-19/England/205280030/2020; 2020-12-24), and EPI_ISL_819798 (hCoV-19/England/OXON-F440A7/2020; 2020-12-18), all as of Jan. 20, 2021; and hCoV-19/Sweden/20-13194/2020 (EPI_ISL_766709), hCoV-19/England/205280030/2020 (EPI_ISL_770441), hCoV-19/France/PAC-NRC2933/2020 (EPI_ISL_768828), hCoV-19/South Korea/KDCA0463/2020 (EPI_ISL_762992), hCoV-19/Japan/IC-0433/2020 (EPUSL_768642), hCoV-19/Australia/NSW3876/2021 (EPI_ISL_775242), hCoV-19/Australia/NSW3872/2021 (EPI_ISL_775245), hCoV-19/France/PAC-NRC2929/2020 (EPUSL_768827), hCoV-19/England/205300109/2020 (EPI_ISL_770467), hCoV-19/England/205320747/2020 (EPI_ISL_770469), hCoV-19/England/205261884/2020 (EPI_ISL_770438), hCoV-19/England/205260233/2020 (EPI_ISL_770437), hCoV-19/England/ALDP-C8FEC7/2020 (EPI_ISL_777292), hCoV-19/England/205221138/2020 (EPI_ISL_766245), hCoV-19/England/205300065/2020 (EPI_ISL_770463), hCoV-19/Botswana/1217-IN1699/2020 (EPI_ISL_770472), hCoV-19/Botswana/1217-IN1660/2020 (EPI_ISL_770471), hCoV-19/England/ALDP-C8E7FA/2020 (EPI_ISL_777266), hCoV-19/England/MILK-C90388/2020 (EPI_ISL_777229), hCoV-19/Botswana/CV1615722/2020 (EPI_ISL_770474), hCoV-19/Botswana/CV1605828/2020 (EPI_ISL_770473), hCoV-19/Scotland/EDB11343/2020 (EPI_ISL_764279), hCoV-19/Scotland/EDB11342/2020 (EPI_ISL_764278), hCoV-19/England/ALDP-C690AF/2020 (EPI_ISL_777190), hCoV-19/Botswana/1223-IN1490/2020 (EPI_ISL_770475), hCoV-19/England/MILK-CA9C09/2020 (EPI_ISL_762362), hCoV-19/England/ALDP-CB4807/2020 (EPUSL_761052), hCoV-19/England/205300064/2020 (EPI_ISL_770462), hCoV-19/England/MILK-CA9BB1/2020 (EPI_ISL_762499), hCoV-19/England/MILK-CAE2B7/2020 (EPUSL_761059), hCoV-19/England/205390867/2021 (EPI_ISL_768815), hCoV-19/Botswana/1224-IN462/2020|(EPI_ISL_770470), hCoV-19/England/205280028/2020 (EPI_ISL_770439), and hCoV-19/England/205280029/2020 (EPI_ISL_770440), all as of Jun. 28, 2021; and all incorporated herein by reference as though fully set forth in their entirety.

Examples of the Gamma (Brazil) variant include but are not limited to GISAID ID Nos. EPI_ISL_677212 (hCoV-19/USANA-DCLS-2187/2020; 2020-11-12), EPI_ISL_723494 (hCoV-19/USANA-DCLS-2191/2020; 2020-11-12), EPI_ISL_845768 (hCoV-19/USA/GA-EHC-458R/2021; 2021-01-05), EPI_ISL_848196 (hCoV-19/Canada/LTRI-1192/2020; 2020-12-24), and EPUSL_848197 (hCoV-19/Canada/LTRI-1258/2020); 2020-12-24), all as of Jan. 20, 2021; and EPI_ISL_792680, EPI_ISL_792681, EPI_ISL_804814, EPI_ISL_804815, EPI_ISL_1468430, EPI_ISL_1483099, EPI_ISL_1483589, and EPI_ISL_1483773, all as of Jun. 28, 2021; and all incorporated herein by reference as though fully set forth in their entirety.

Examples of the Delta (B1.617.2) variant include but are not limited to GISAID ID Nos. EPI_ISL_1653403, EPI_ISL_1697977, EPI_ISL_1718959, EPI_ISL_1719027, EPI_ISL_2121225, EPI_ISL_2121637, EPI_ISL_2121989, EPI_ISL_2122659, EPI_ISL_2125463, EPI_ISL_2126212, EPI_ISL_2126374, EPI_ISL_2127610, EPI_ISL_2127624, EPI_ISL_2127831, and EPI_ISL_2131345, all as of Jun. 28, 2021.

TABLE 1
Primers for SARS-COV-2
Primers for RT-PCR

| SEQ | | | |
|---|---|---|---|
| ID | | | |
| NO: | No. | Name | oligo sequence 5′-3′ |
| 3 | 2312 | 2312-Fr1-T7G-F3 | GAtaatacgactcactatagATTAAAGGTTTATACCTTCCCAGGTAAC |
| 4 | 1786 | 1786-COV-2 | GATGCCAAAATAATGGCGATCTC |
| 5 | 1787 | 1787-COV-3 | GTTGGTTGCCATAACAAGTGTG |
| 6 | 1788 | 1788-COV-4 | CTAATTGAGGTTGAACCTCAACAATTG |
| 7 | 1789 | 1789-COV-5 | GAGTATGGTACTGAAGATGATTACCAAG |
| 8 | 1790 | 1790-COV-6 | CTAGGTGGAATGTGGTAGGATTAC |
| 9 | 1791 | 1791-COV-7 | GCTGTTACAGCGTATAATGGTTATCTTAC |
| 10 | 1792 | 1792-COV-8 | GCTGGTTTAAGTATAATGTCTCCTACAAC |
| 11 | 1793 | 1793-COV-9 | GCACAAAACCAGTTGAAACATCAAATTC |
| 12 | 1794 | 1794-COV-10 | GCAACTAGTGTTTTGAGTTTTTCCATTG |
| 13 | 1795 | 1795-COV-11 | GTGAAGAATCATCTGCAAAATCAGC |
| 14 | 1796 | 1796-COV-12 | CAAATGATATAAGCAATTGTTATCCAGAAAGG |
| 15 | 1797 | 1797-COV-13 | GCCTTTAATACTTTACTATTCCTTATGTCATTCAC |
| 16 | 1798 | 1798-COV-14 | CCAGACAAACTAGTATCAACCATATCC |
| 17 | 1799 | 1799-COV-15 | GCTATGGGTATTATTGCTATGTCTG |
| 18 | 1800 | 1800-COV-16 | CCTACAAGGTGGTTCCAGTTC |
| 19 | 1801 | 1801-COV-17 | CGACAGATGTCTTGTGCTG |
| 20 | 1802 | 1802-COV-18 | GGTATCCAGTTGAAACTACAAATGG |
| 21 | 1803 | 1803-COV-19 | GATCAGACATACCACCCAAATTG |
| 22 | 1804 | 1804-COV-20 | CTTATGTATTGTAAGTACAAATGAAAGACATCAG |
| 23 | 1805 | 1805-COV-21 | GGTGATGATTATGTGTACCTTCCTTAC |
| 24 | 1806 | 1806-COV-22 | CTGTTAATTGCAGATGAAACATCATGC |
| 25 | 1807 | 1807-COV-23 | GTGTGTAGACTTATGAAAACTATAGGTCC |
| 26 | 1808 | 1808-COV-24 | CATACAAACTGCCACCATCAC |
| 27 | 1809 | 1809-COV-25 | CCTTGTAGTGACAAAGCTTATAAAATAGAAG |
| 28 | 1810 | 1810-COV-26 | CTGGTGCAACTCCTTTATCAG |
| 29 | 1811 | 1811-COV-27 | GCAAAGAATGCTATTAGAAAAGTGTGAC |
| 30 | 1812 | 1812-COV-28 | GATAGATTCCTTTTTCTACAGTGAAGGATTTC |
| 31 | 1813 | 1813-COV-29 | GACTCCTGGTGATTCTTCTTCAG |
| 32 | 1814 | 1814-COV-30 | CTCTAGCAGCAATATCACCAAGG |
| 33 | 1815 | 1815-COV-31 | GCACAAGTCAAACAAATTTACAAAACAC |
| 34 | 1816 | 1816-COV-32 | CAAAAGGTGTGAGTAAACTGTTACAAAC |
| 35 | 1817 | 1817-COV-33 | CTCACTCCCTTTCGGATGG |
| 36 | 1818 | 1818-COV-34 | GAGGTTTATGATGTAATCAAGATTCCAAATGG |
| 37 | 1819 | 1819-COV-35 | GCTACAGGATTGGCAACTATAAATTAAAC |
| 38 | 1820 | 1820-COV-36 | CCATTCTAGCAGGAGAAGTTCC |
| 39 | 1821 | 1821-COV-37 | GCAATCCTGCTAACAATGCTG |
| 40 | 1822 | 1822-COV-38 | ttttTTTTTTTTTTTTTTTTTTTTTGTCATTCTCCTAAGAAGCTATTAA |
| | | | AATC |

Sequences

SEQ ID NO: 1 (deoptimized in reference to Washington isolate (GenBank: MN985325.1),
with a 36 nucleotide deletion in the spike protein and without a polyA tail):
attaaaggtttataccttcccaggtaacaaaccaaccaactttcgatctcttgtagatctgttctctaaacgaactttaaaatctgtgtggctg
tcactcggctgcatgcttagtgcactcacgcagtataattaataactaattactgtcgttgacaggacacgagtaactcgtctatcttctgcaggctgcttacg
gtttcgtccgtgttgcagccgatcatcagcacatctaggtttcgtccgggtgtgaccgaaaggtaagatggagagccttgtccctggtttcaacgagaaaa
cacacgtccaactcagtttgcctgttttacaggttcgcgacgtgctcgtacgtggctttggagactccgtggaggaggtcttatcagaggcacgtcaacatc
ttaaagatggcacttgtggcttagtagaagttgaaaaaggcgttttgcctcaacttgaacagccctatgtgttcatcaaacgttcggatgctcgaactgcacct
catggtcatgttatggttgagctggtagcagaactcgaaggcattcagtacggtcgtagtggtgagacacttggtgtccttgtccctcatgtgggcgaaata
ccagtggcttaccgcaaggttcttcttcgtaagaacggtaataaaggagctggtggccatagttacggcgccgatctaaagtcatttgacttaggcgacga
gcttggcactgatccttatgaagattttcaagaaaactggaacactaaacatagcagtggtgttacccgtgaactcatgcgtgagcttaacggaggggcat
acactcgctatgtcgataacaacttctgtggccctgatggctaccctcttgagtgcattaaagaccttctagcacgtgctggtaaagcttcatgcactttgtcc
gaacaactggactttattgacactaagaggggtgtatactgctgccgtgaacatgagcatgaaattgcttggtacacggaacgttctgaaaagagctatga
attgcagacaccttttgaaattaaattggcaaagaaatttgacaccttcaatggggaatgtccaaattttgtatttcccttaaattccataatcaagactattcaac
caagggttgaaaagaaaaagcttgatggctttatgggtagaattcgatctgtctatccagttgcgtcaccaaatgaatgcaaccaaatgtgcctttcaactct
catgaagtgtgatcattgtggtgaaacttcatggcagacgggcgattttgttaaagccacttgcgaattttgtggcactgagaatttgactaaagaaggtgcc
actacttgtggttacttaccccaaaatgctgttgttaaaatttattgtccagcatgtcacaattcagaagtaggacctgagcatagtcttgccgaataccataat
gaatctggcttgaaaaccattcttcgtaagggtggtcgcactattgcctttggaggctgtgtgttctcttatgttggttgccataacaagtgtgcctattgggttc
cacgtgctagcgctaacataggttgtaaccatacaggtgttgttggagaaggttccgaaggtcttaatgacaaccttcttgaaatactccaaaaagagaaag
tcaacatcaatattgttggtgactttaaacttaatgaagagatcgccattattttggcatctttttctgcttccacaagtgcttttgtggaaactgtgaaaggtttgg
attataaagcattcaaacaaattgttgaatcctgtggtaattttaaagttacaaaaggaaaagctaaaaaaggtgcctggaatattggtgaacagaaatcaat
actgagtcctctttatgcatttgcatcagaggctgctcgtgttgtacgatcaattttctcccgcactcttgaaactgctcaaaattctgtgcgtgttttacagaag
gccgctataacaatactagatggaatttcacagtattcactgagactcattgatgctatgatgttcacatctgatttggctactaacaatctagttgtaatggcct
acattacaggtggtgttgttcagttgacttcgcagtggctaactaacatctttggcactgtttatgaaaaactcaaacccgtccttgattggcttgaagagaagt
ttaaggaaggtgtagagtttcttagagacggttgggaaattgttaaatttatctcaacctgtgcttgtgaaattgtcggtggacaaattgtcacctgtgcaaag
gaaattaaggagagtgttcagacattctttaagcttgtaaataaatttttggctttgtgtgctgactctatcattattggtggagctaaacttaaagccttgaattta
ggtgaaacatttgtcacgcactcaaagggattgtacagaaagtgtgttaaatccagagaagaaactggcctactcatgcctctaaaagccccaaaagaaat
tatcttcttagagggagaaacacttcccacagaagtgttaacagaggaagttgtcttgaaaactggtgatttacaaccattagaacaacctactagtgaagct
gttgaagctccattggttggtacaccagtttgtattaacgggcttatgttgctcgaaatcaaagacacagaaaagtactgtgcccttgcacctaatatgatggt
aacaaacaataccttcacactcaaaggcggtgcaccaacaaaggttacttttggtgatgacactgtgatagaagtgcaaggttacaagagtgtgaatatca
cttttgaacttgatgaaaggattgataaagtacttaatgagaagtgctctgcctatacagttgaactcggtacagaagtaaatgagttcgcctgtgttgtggca
gatgctgtcataaaaactttgcaaccagtatctgaattacttacaccactgggcattgatttagatgagtggagtatggctacatactacttatttgatgagtctg
gtgagtttaTattggcttcacatatgtattgttctttctaccctccagatgaggatgaagaagaaggtgattgtgaagaagaagagtttgagccatcaactca
atatgagtatggtactgaagatgattaccaaggtaaacctttggaatttggtgccacttctgctgctcttcaacctgaagaagagcaagaagaagattggtta
gatgatgatagtcaacaaactgttggtcaacaagacggcagtgaggacaatcagacaactactattcaaacaattgttgaggttcaacctcaattagagatg
gaacttacaccagttgttcagactattgaagtgaatagttttagtggttatttaaaacttactgacaatgtatacattaaaaatgcagacattgtggaagaagcta
aaaaggtaaaaccaacagtggttgttaatgcagccaatgtttaccttaaacatggaggaggtgttgcaggagccttaaataaggctactaacaatgccatg
caagttgaatctgatgattacatagctactaatggaccacttaaagtgggtggtagttgtgttttaagcggacacaatcttgctaaacactgtcttcatgttgtc
ggcccaaatgttaacaaaggtgaagacattcaacttcttaagagtgcttatgaaaattttaatcagcacgaagttctacttgcaccattattatcagctggtattt
ttggtgctgaccctatacattctttaagagtttgtgtagatactgttcgcacaaatgtctacttagctgtctttgataaaaatctctatgacaaacttgtttcaagctt
tttggaaatgaagagtgaaaagcaagttgaacaaaagatcgctgagattcctaaagaggaagttaagccatttataactgaaagtaaaccttcagttgaaca
gagaaaacaagatgataagaaaatcaaagcttgtgttgaagaagttacaacaactctggaagaaactaagttcctcacagaaaacttgttactttatattgac
attaatggcaatcttcatccagattctgccactcttgttagtgacattgacatcactttcttaaagaaagatgctccatatatagtgggtgatgttgttcaagagg
gtgttttaactgctgtggttatacctactaaaaaggctggtggcactactgaaatgctagcgaaagctttgagaaaagtgccaacagacaattatataaccac
ttacccgggtcagggtttaaatggttacactgtagaggaggcaaagacagtgcttaaaaagtgtaaaagtgccttttacattctaccatctattatctctaatga
gaagcaagaaattcttggaactgtttcttggaatttgcgagaaatgcttgcacatgcagaagaaacacgcaaattaatgcctgtctgtgtggaaactaaagc
catagtttcaactatacagcgtaaatataagggtattaaaatacaagagggtgtggttgattatggtgctagattttacttttacaccagtaaaacaactgtagc
gtcacttatcaacacacttaacgatctaaatgaaactcttgttacaatgccacttggctatgtaacacatggcttaaatttggaagaagctgctcggtatatgag
atctctcaaagtgccagctacagtttctgtttcttcacctgatgctgttacagcgtataatggttatcttacttcttcttctaaaacacctgaagaacattttattgaa
accatctcacttgctggttcctataaagattggtcctattctggacaatctacacaactaggtatagaatttcttaagagaggtgataaaagtgtatattacacta
gtaatcctaccacattccacctagatggtgaagttatcacctttgacaatcttaagacacttctttctttgagagaagtgaggactattaaggtgtttacaacagt
agacaacattaacctccacacgcaagttgtggacatgtcaatgacatatggacaacagtttggtccaacttatttggatggagctgatgttactaaaataaaa
cctcataattcacatgaaggtaaaacattttatgttttacctaatgatgacactctacgtgttgaggcttttgagtactaccacacaactgatcctagttttctggg
taggtacatgtcagcattaaatcacactaaaaagtggaaatacccacaagttaatggtttaacttctattaaatgggcagataacaactgttatcttgccactg
cattgttaacactccaacaaatagagttgaagtttaatccacctgctctacaagatgcttattacagagcaagggctggtgaagctgctaacttttgtgcactt
atcttagcctactgtaataagacagtaggtgagttaggtgatgttagagaaacaatgagttacttgtttcaacatgccaatttagattcttgcaaaagagtcttg
aacgtggtgtgtaaaacttgtggacaacagcagacaacccttaagggtgtagaagctgttatgtacatgggcacactttcttatgaacaatttaagaaaggt
gttcagataccttgtacgtgtggtaaacaagctacaaaatatctagtacaacaggagtcaccttttgttatgatgtcagcaccacctgctcagtatgaacttaa
gcatggtacatttacttgtgctagtgagtacactggtaattaccagtgtggtcactataaacatataacttctaaagaaactttgtattgcatagacggtgcttta
cttacaaagtcctcagaatacaaaggtcctattacggatgttttctacaaagaaaacagttacacaacaaccataaaaccagttacttataaattggatggtgt
tgtttgtacagaaattgaccctaagttggacaattattataagaaagacaattcttatttcacagagcaaccaattgatcttgtaccaaaccaaccatatccaaa
cgcaagcttcgataattttaagtttgtatgtgataatatcaaatttgctgatgatttaaaccagttaactggttataagaaacctgcttcaagagagcttaaagtta
catttttccctgacttaaatggtgatgtggtggctattgattataaacactacacaccctcttttaagaaaggagctaaattgttacataaacctattgtttggcat
gttaacaatgcaactaataaagccacgtataaaccaaatacctggtgtatacgttgtctttggagcacaaaaccagttgaaacatcaaattcgtttgatgtact
gaagtcagaggacgcgcagggaatggataatcttgcctgcgaagatctaaaaccagtctctgaagaagtagtggaaaatcctaccatacagaaagacgt
tcttgagtgtaatgtgaaaactaccgaagttgtaggagacattatacttaaaccagcaaataatagtttaaaaattacagaagaggttggccacacagatcta
atggctgcttatgtagacaattctagtcttactattaagaaacctaatgaattatctagagtattaggtttgaaaacccttgctactcatggtttagctgctgttaat
agtgtcccttgggatactatagctaattatgctaagccttttcttaacaaagttgttagtacaactactaacatagttacacggtgtttaaaccgtgtttgtactaat
tatatgccttatttctttactttattgctacaattgtgtacttttactagaagtacaaattctagaattaaagcatctatgccgactactatagcaaagaatactgtta
agagtgtcggtaaattttgtctagaggcttcatttaattatttgaagtcacctaatttttctaaactgataaatattataatttggtttttactattaagtgtttgcctag
gttctttaatctactcaaccgctgctttaggtgttttaatgtctaatttaggcatgccttcttactgtactggttacagagaaggctatttgaactctactaatgtcac
tattgcaacctactgtactggttctataccttgtagtgtttgtcttagtggtttagattctttagacacctatccttctttagaaactatacaaattaccatttcatctttt
aaatgggatttaactgcttttggcttagttgcagagtggtttttggcatatattcttttcactaggtttttctatgtacttggattggctgcaatcatgcaattgtttttc
agctattttgcagtacattttattagtaattcttggcttatgtggttaataattaatcttgtacaaatggccccgatttcagctatggttagaatgtacatcttctttgc
atcattttattatgtatggaaaagttatgtgcatgttgtagacggttgtaattcatcaacttgtatgatgtgttacaaacgtaatagagcaacaagagtcgaatgt
acaactattgttaatggtgttagaaggtccttttatgtctatgctaatggaggtaaaggcttttgcaaactacacaattggaattgtgttaattgtgatacattctgt
gctggtagtacatttattagtgatgaagttgcgagagacttgtcactacagtttaaaagaccaataaatcctactgaccagtcttcttacatcgttgatagtgtta
cagtgaagaatggttccatccatctttactttgataaagctggtcaaaagacttatgaaagacattctctctctcattttgttaacttagacaacctgagagctaa
taacactaaaggttcattgcctattaatgttatagtttttgatggtaaatcaaaatgtgaagaatcatctgcaaaatcagcgtctgtttactacagtcagcttatgt
gtcaacctatactgttactagatcaggcattagtgtctgatgttggtgatagtgcggaagttgcagttaaaatgtttgatgcttacgttaatacgttttcatcaact
tttaacgtaccaatggaaaaactcaaaacactagttgcaactgcagaagctgaacttgcaaagaatgtgtccttagacaatgtcttatctacttttatttcagca
gctcggcaagggtttgttgattcagatgtagaaactaaagatgttgttgaatgtcttaaattgtcacatcaatctgacatagaagttactggcgatagttgtaat
aactatatgctcacctataacaaagttgaaaacatgacaccccgtgaccttggtgcttgtattgactgtagtgcgcgtcatattaatgcgcaggtagcaaaaa
gtcacaacattgctttgatatggaacgttaaagatttcatgtcattgtctgaacaactacgaaaacaaatacgtagtgctgctaaaaagaataacttacctttta
agttgacatgtgcaactactagacaagttgttaatgttgtaacaacaaagatagcacttaagggtggtaaaattgttaataattggttgaagcagttaattaaa
gttacacttgtgttcctttttgttgctgctattttctatttaataacacctgttcatgtcatgtctaaacatactgacttttcaagtgaaatcataggatacaaggctatt
gatggtggtgtcactcgtgacatagcatctacagatacttgttttgctaacaaacatgctgattttgacacatggtttagtcagcgtggtggtagttatactaatg
acaaagcttgcccattgattgctgcagtcataacaagagaagtgggttttgtcgtgcctggtttgcctggcacgatattacgcacaactaatggtgactttttg
catttcttacctagagtttttagtgcagttggtaacatctgttacacaccatcaaaacttatagagtacactgactttgcaacatcagcttgtgttttggctgctga
atgtacaatttttaaagatgcttctggtaagccagtaccatattgttatgataccaatgtactagaaggttctgttgcttatgaaagtttacgccctgacacacgtt
atgtgctcatggatggctctattattcaatttcctaacacctaccttgaaggttctgttagagtggtaacaacCtttgattctgagtactgtaggcacggcactt
gtgaaagatcagaagctggtgtttgtgtatctactagtggtagatgggtacttaacaatgattattacagatctttaccaggagttttctgtggtgtagatgctgt
aaatttacttactaatatgtttacaccactaattcaacctattggtgctttggacatatcagcatctatagtagctggtggtattgtagctatcgtagtaacatgcct
tgcctactattttatgaggtttagaagagcttttggtgaatacagtcatgtagttgcctttaatactttactattccttatgtcattcactgtactctgtttaacaccag
tttactcattcttacctggtgtttattctgttatttacttgtacttgacattttatcttactaatgatgtttcttttttagcacatattcagtggatggttatgttcacaccttt
agtacctttctggataacaattgcttatatcatttgtatttccacaaagcatttctattggttctttagtaattacctaaagagacgtgtagtctttaatggtgtttcctt
tagtacttttgaagaagctgcgctgtgcacctttttgttaaataaagaaatgtatctaaagttgcgtagtgatgtgctattacctcttacgcaatataatagatact
tagctctttataataagtacaagtattttagtggagcaatggatacaactagctacagagaagctgcttgttgtcatctcgcaaaggctctcaatgacttcagta
actcaggttctgatgttctttaccaaccaccacaaacctctatcacctcagctgttttgcagagtggttttagaaaaatggcattcccatctggtaaagttgagg
gttgtatggtacaagtaacttgtggtacaactacacttaacggtctttggcttgatgacgtagtttactgtccaagacatgtgatctgcacctctgaagacatgc
ttaaccctaattatgaagatttactcattcgtaagtctaatcataatttcttggtacaggctggtaatgttcaactcagggttattggacattctatgcaaaattgtg
tacttaagcttaaggttgatacagccaatcctaagacacctaagtataagtttgttcgcattcaaccaggacagactttttcagtgttagcttgttacaatggttc
accatctggtgtttaccaatgtgctatgaggcccaatttcactattaagggttcattccttaatggttcatgtggtagtgttggttttaacatagattatgactgtgt
ctctttttgttacatgcaccatatggaattaccaactggagttcatgctggcacagacttagaaggtaacttttatggaccttttgttgacaggcaaacagcaca
agcagctggtacggacacaactattacagttaatgttttagcttggttgtacgctgctgttataaatggagacaggtggtttctcaatcgatttaccacaactctt
aatgactttaaccttgtggctatgaagtacaattatgaacctctaacacaagaccatgttgacatactaggacctctttctgctcaaactggaattgccgtttta
gatatgtgtgcttcattaaaagaattactgcaaaatggtatgaatggacgtaccatattgggtagtgctttattagaagatgaatttacaccttttgatgttgttag
acaatgctcaggtgttactttccaaagtgcagtgaaaagaacaatcaagggtacacaccactggttgttactcacaattttgacttcacttttagttttagtcca
gagtactcaatggtctttgttcttttttttgtatgaaaatgcctttttaccttttgctatgggtattattgctatgtctgcttttgcaatgatgtttgtcaaacataagcat
gcatttctctgtttgtttttgttaccttctcttgccactgtagcttattttaatatggtctatatgcctgctagttgggtgatgcgtattatgacatggttggatatggtt
gatactagtttgtctggttttaagctaaaagactgtgttatgtatgcatcagctgtagtgttactaatccttatgacagcaagaactgtgtatgatgatggtgcta
ggagagtgtggacacttatgaatgtcttgacactcgtttataaagtttattatggtaatgctttagatcaagccatttccatgtgggctcttataatctctgttactt
ctaactactcaggtgtagttacaactgtcatgttCttggccagaggtattgtttttatgtgtgttgagtattgccctattttcttcataactggtaatacacttcagtg
tataatgctagtttattgtttcttaggctatttttgtacttgttactttggcctcttttgtttactcaaccgctactttagactgactcttggtgtttatgattacttagtttct
acacaggagtttagatatatgaattcacagggactactcccacccaagaatagcatagatgccttcaaactcaacattaaattgttgggtgttggtggcaaa
ccttgtatcaaagtagccactgtacagtctaaaatgtcagatgtaaagtgcacatcagtagtcttactctcagttttgcaacaactcagagtagaatcatcatct
aaattgtgggctcaatgtgtccagttacacaatgacattctcttagctaaagatactactgaagcctttgaaaaaatggtttcactactttctgttttgctttccatg
cagggtgctgtagacataaacaagctttgtgaagaaatgctggacaacagggcaaccttacaagctatagcctcagagtttagttcccttccatcatatgca
gcttttgctactgctcaagaagcttatgagcaggctgttgctaatggtgattctgaagttgttcttaaaaagttgaagaagtctttgaatgtggctaaatctgaat
ttgaccgtgatgcagccatgcaacgtaagttggaaaagatggctgatcaagctatgacccaaatgtataaacaggctagatctgaggacaagagggcaa
aagttactagtgctatgcagacaatgcttttcactatgcttagaaagttggataatgatgcactcaacaacattatcaacaatgcaagagatggttgtgttccct
tgaacataatacctcttacaacagcagccaaactaatggttgtcataccagactataacacatataaaaatacgtgtgatggtacaacatttacttatgcatca
gcattgtgggaaatccaacaggttgtagatgcagatagtaaaattgttcaacttagtgaaattagtatggacaattcacctaatttagcatggcctcttattgta
acagctttaagggccaattctgctgtcaaattacagaataatgagcttagtcctgttgcactacgacagatgtcttgtgctgccggtactacacaaactgcttg
cactgatgacaatgcgttagcttactacaacacaacaaagggaggtaggtttgtacttgcactgttatccgatttacaggatttgaaatgggctagattcccta
agagtgatggaactggtactatctatacagaactggaaccaccttgtaggtttgttacagacacacctaaaggtcctaaagtgaagtatttatactttattaaa
ggattaaacaacctaaatagaggtatggtacttggtagtttagctgccacagtacgtctacaagctggtaatgcaacagaagtgcctgccaattcaactgtat
tatctttctgtgcttttgctgtagatgctgctaaagcttacaaagattatctagctagtgggggacaaccaatcactaattgtgttaagatgttgtgtacacacac
tggtactggtcaggcaataacagttacaccggaagccaatatggatcaagaatcctttggtggtgcatcgtgttgtctgtactgccgttgccacatagatcat
ccaaatcctaaaggattttgtgacttaaaaggtaagtatgtacaaatacctacaacttgtgctaatgaccctgtgggttttacacttaaaaacacagtctgtacc
gtctgcggtatgtggaaaggttatggctgtagttgtgatcaactccgcgaacccatgcttcagtcagctgatgcacaatcgtttttaaacgggtttgcggtgta
agtgcagcccgtcttacaccgtgcggcacaggcactagtactgatgtcgtatacagggcttttgacatctacaatgataaagtagctggttttgctaaattcct
aaaaactaattgttgtcgcttccaagaaaaggacgaagatgacaatttaattgattcttactttgtagttaagagacacactttctctaactaccaacatgaaga
aacaatttataatttacttaaggattgtccagctgttgctaaacatgacttctttaagtttagaatagacggtgacatggtaccacatatatcacgtcaacgtctta
ctaaatacacaatggcagacctcgtctatgctttaaggcattttgatgaaggtaattgtgacacattaaaagaaatacttgtcacatacaattgttgtgatgatg
attatttcaataaaaaggactggtatgattttgtagaaaacccagatatattacgcgtatacgccaacttaggtgaacgtgtacgccaagctttgttaaaaaca
gtacaattctgtgatgccatgcgaaatgctggtattgttggtgtactgacattagataatcaagatctcaatggtaactggtatgatttcggtgatttcatacaaa
ccacgccaggtagtggagttcctgttgtagattcttattattcattgttaatgcctatattaaccttgaccagggctttaactgcagagtcacatgttgacactga
cttaacaaagccttacattaagtgggatttgttaaaatatgacttcacggaagagaggttaaaactctttgaccgttattttaaatattgggatcagacatacca
cccaaattgtgttaactgtttggatgacagatgcattctgcattgtgcaaactttaatgttttattctctacagtgttcccacctacaagttttggaccactagtga
gaaaaatatttgttgatggtgttccatttgtagtttcaactggataccacttcagagagctaggtgttgtacataatcaggatgtaaacttacatagctctagact
tagttttaaggaattacttgtgtatgctgctgaccctgctatgcacgctgcttctggtaatctattactagataaacgcactacgtgcttttcagtagctgcactta
ctaacaatgttgcttttcaaactgtcaaacccggtaattttaacaaagacttctatgactttgctgtgtctaagggtttctttaaggaaggaagttctgttgaatta
aaacacttcttctttgctcaggatggtaatgctgctatcagcgattatgactactatcgttataatctaccaacaatgtgtgatatcagacaactactatttgtagt
tgaagttgttgataagtactttgattgttacgatggtggctgtattaatgctaaccaagtcatcgtcaacaacctagacaaatcagctggttttccatttaataaat
ggggtaaggctagactttattatgattcaatgagttatgaggatcaagatgcacttttcgcatatacaaaacgtaatgtcatccctactataactcaaatgaatc
ttaagtatgccattagtgcaaagaatagagctcgcaccgtagctggtgtctctatctgtagtactatgaccaatagacagtttcatcaaaaattattgaaatcaa
tagccgccactagaggagctactgtagtaattggaacaagcaaattctatggtggttggcacaacatgttaaaaactgtttatagtgatgtagaaaaccctca
ccttatgggttgggattatcctaaatgtgatagagccatgcctaacatgcttagaattatggcctcacttgttcttgctcgcaaacatacaacgtgttgtagcttg
tcacaccgtttctatagattagctaatgagtgtgctcaagtattgagtgaaatggtcatgtgtggcggttcactatatgttaaaccaggtggaacctcatcagg
agatgccacaactgcttatgctaatagtgtttttaacatttgtcaagctgtcacggccaatgttaatgcacttttatctactgatggtaacaaaattgccgataag
tatgtccgcaatttacaacacagactttatgagtgtctctatagaaatagagatgttgacacagactttgtgaatgagttttacgcatatttgcgtaaacatttctc
aatgatgatactctctgacgatgctgttgtgtgtttcaatagcacttatgcatctcaaggtctagtggctagcataaagaactttaagtcagttctttattatcaaa
acaatgtttttatgtctgaagcaaaatgttggactgagactgaccttactaaaggacctcatgaattttgctctcaacatacaatgctagttaaacagggtgatg
attatgtgtaccttccttacccagatccatcaagaatcctaggggccggctgttttgtagatgatatcgtaaaaacagatggtacacttatgattgaacggttc
gtgtctttagctatagatgcttacccacttactaaacatcctaatcaggagtatgctgatgtctttcatttgtacttacaatacataagaaagctacatgatgagtt
aacaggacacatgttagacatgtattctgttatgcttactaatgataacacttcaaggtattgggaacctgagttttatgaggctatgtacacaccgcatacagt
cttacaggctgttggggcttgtgttctttgcaattcacagacttcattaagatgtggtgcttgcatacgtagaccattcttatgttgtaaatgctgttacgaccatg
tcatatcaacatcacataaattagtcttgtctgttaatccgtatgtttgcaGtgctccaggttgtgatgtcacagatgtgactcaactttacttaggaggtatgag
ctattattgtaaatcacataaaccacccattagttttccattgtgtgctaatggacaagtttttggtttatataaaaatacatgtgttggtagcgataatgttactga
ctttaatgcaattgcaacatgtgactggacaaatgctggtgattacattttagctaacacctgtactgaaagactcaagctttttgcagcagaaacgctcaaag
ctactgaggagacatttaaactgtcttatggtattgctactgtacgtgaagtgctgtctgacagagaattacatctttcatgggaagttggtaaacctagacca
ccacttaaccgaaattatgtctttactggttatcgtgtaactaaaaacagtaaagtacaaataggagagtacacctttgaaaaaggtgactatggtgatgctgt
tgtttaccgaggtacaacaacttacaaattaaatgttggtgattattttgtgctgacatcacatacagtaatgccattaagtgcacctacactagtgccacaaga
gcactatgttagaattactggcttatacccaacactcaatatctcagatgagttttctagcaatgttgcaaattatcaaaaggttggtatgcaaaagtattctaca
ctccagggaccacctggtactggtaagagtcattttgctattggcctagctctctactacccttctgctcgcatagtgtatacagcttgctctcatgccgctgtt
gatgcactatgtgagaaggcattaaaatatttgcctatagataaatgtagtagaattatacctgcacgtgctcgtgtagagtgttttgataaattcaaagtgaatt
caacattagaacagtatgtcttttgtactgtaaatgcattgcctgagacgacagcagatatagttgtctttgatgaaatttcaatggccacaaattatgatttgag
tgttgtcaatgccagattacgtgctaagcactatgtgtacattggcgaccctgctcaattacctgcaccacgcacattgctaactaagggcacactagaacc
agaatatttcaattcagtgtgtagacttatgaaaactataggtccagacatgttcctcggaacttgtcggcgttgtcctgctgaaattgttgacactgtgagtgc
tttggtttatgataataagcttaaagcacataaagacaaatcagctcaatgctttaaaatgttttataagggtgttatcacgcatgatgtttcatctgcaattaaca
ggccacaaataggcgtggtaagagaattccttacacgtaaccctgcttggagaaaagctgtctttatttcaccttataattcacagaatgctgtagcctcaaa
gattttgggactaccaactcaaactgttgattcatcacagggctcagaatatgactatgtcatattcactcaaaccactgaaacagctcactcttgtaatgtaaa
cagatttaatgttgctattaccagagcaaaagtaggcatactttgcataatgtctgatagagacctttatgacaagttgcaatttacaagtcttgaaattccacgt
aggaatgtggcaactttacaagctgaaaatgtaacaggactttttaaagattgtagtaaggtaatcactgggttacatcctacacaggcacctacacacctca
gtgttgacactaaattcaaaactgaaggtttatgtgttgacatacctggcatacctaaggacatgacctatagaagactcatctctatgatgggttttaaaatga
attatcaagttaatggttaccctaacatgtttatcacccgcgaagaagctataagacatgtacgtgcatggattggcttcgatgtcgaggggtgtcatgctact
agagaagctgttggtaccaatttacctttacagctaggtttttctacaggtgttaacctagttgctgtacctacaggttatgttgatacacctaataatacagattt
ttccagagttagtgctaaaccaccgcctggagatcaatttaaacacctcataccacttatgtacaaaggacttccttggaatgtagtgcgtataaagattgtac
aaatgttaagtgacacacttaaaaatctctctgacagagtcgtatttgtcttatgggcacatggctttgagttgacatctatgaagtattttgtgaaaataggacc
tgagcgcacctgttgtctatgtgatagacgtgccacatgcttttccactgcttcagacacttatgcctgttggcatcattctattggatttgattacgtctataatc
cgtttatgattgatgttcaacaatggggttttacaggtaacctacaaagcaaccatgatctgtattgtcaagtccatggtaatgcacatgtagctagttgtgatg
caatcatgactaggtgtctagctgtccacgagtgctttgttaagcgtgttgactggactattgaatatcctataattggtgatgaactgaagattaatgcggctt
gtagaaaggttcaacacatggttgttaaagctgcattattagcagacaaattcccagttcttcacgacattggtaaccctaaagctattaagtgtgtacctcaa
gctgatgtagaatggaagttctatgatgcacagccttgtagtgacaaagcttataaaatagaagaattattctattcttatgccacacattctgacaaattcaca
gatggtgtatgcctattttggaattgcaatgtcgatagatatcctgctaattccattgtttgtagatttgacactagagtgctatctaaccttaacttgcctggttgt
gatggtggcagtttgtatgtaaataaacatgcattccacacaccagcttttgataaaagtgcttttgttaatttaaaacaattaccatttttctattactctgacagt
ccatgtgagtctcatggaaaacaagtagtgtcagatatagattatgtaccactaaagtctgctacgtgtataacacgttgcaatttaggtggtgctgtctgtag
acatcatgctaatgagtacagattgtatctcgatgcttataacatgatgatctcagctggctttagcttgtgggtttacaaacaatttgatacttataacctctgga
acacttttacaagacttcagagtttagaaaatgtggcttttaatgttgtaaataagggacactttgatggacaacagggtgaagtaccagtttctatcattaata
acactgtttacacaaaagttgatggtgttgatgtagaattgtttgaaaataaaacaacattacctgttaatgtagcatttgagctttgggctaagcgcaacatta
aaccagtaccagaggtgaaaatactcaataatttgggtgtggacattgctgctaatactgtgatctgggactacaaaagagatgctccagcacatatatcta
ctattggtgtttgttctatgactgacatagccaagaaaccaactgaaacgatttgtgcaccactcactgtcttttttgatggtagagttgatggtcaagtagactt
atttagaaatgcccgtaatggtgttcttattacagaaggtagtgttaaaggtttacaaccatctgtaggtcccaaacaagctagtcttaatggagtcacattaat
tggagaagccgtaaaaacacagttcaattattataagaaagttgatggtgttgtccaacaattacctgaaacttactttactcagagtagaaatttacaagaatt
taaacccaggagtcaaatggaaattgatttcttagaattagctatggatgaattcattgaacggtataaattagaaggctatgccttcgaacatatcgtttatgg
agattttagtcatagtcagttaggtggtttacatctactgattggactagctaaacgttttaaggaatcaccttttgaattagaagattttattcctatggacagtac
agttaaaaactatttcataacagatgcgcaaacaggttcatctaagtgtgtgtgttctgttattgatttattacttgatgattttgttgaaataataaaatcccaaga
tttatctgtagtttctaaggttgtcaaagtgactattgactatacagaaatttcatttatgctttggtgtaaagatggccatgtagaaacattttacccaaaattaca
atctagtcaagcgtggcaaccgggtgttgctatgcctaatctttacaaaatgcaaagaatgctattagaaaagtgtgaccttcaaaattatggtgatagtgca
acattacctaaaggcataatgatgaatgtcgcaaaatatactcaactgtgtcaatatttaaacacattaacattagctgtaccctataatatgagagttatacatt
ttggtgctggttctgataaaggagttgcaccaggtacagctgttttaagacagtggttgcctacgggtacgctgcttgtcgattcagatcttaatgactttgtct
ctgatgcagattcaactttgattggtgattgtgcaactgtacatacagctaataaatgggatctcattattagtgatatgtacgaccctaagactaaaaatgttac
aaaagaaaatgactctaaagagggttttttcacttacatttgtgggtttatacaacaaaagctagctcttggaggttccgtggctataaagataacagaacatt
cttggaatgctgatctttataagctcatgggacacttcgcatggtggacagcctttgttactaatgtgaatgcgtcatcatctgaagcatttttaattggatgtaat
tatcttggcaaaccacgcgaacaaatagatggttatgtcatgcatgcaaattacatattttggaggaatacaaatccaattcagttgtcttcctattctttatttga
catgagtaaatttccccttaaattaaggggtactgctgttatgtctttaaaagaaggtcaaatcaatgatatgattttatctcttcttagtaaaggtagacttataat
tagagaaaacaacagagttgttatttctagtgatgttcttgttaacaactaaacgaacaatgtttgtttttcttgttttattgccactagtctctagtcagtgtgttaat
cttacaaccagaactcaattaccccctgcatacactaattctttcacacgtggtgtttattaccctgacaaagttttcagatcctcagttttacattcaactcagga
cttgttcttacctttcttttccaatgttacttggttccatgctatacatgtctctgggaccaatggtactaagaggtttgataaccctgtcctaccatttaatgatggt
gtttattttgcttccaTtgagaagtctaacataataagaggctggatttttggtactactttagattcgaagacccagtccctacttattgttaataacgctactaa
tgttgttattaaagtctgtgaatttcaattttgtaatgatccatttttgggtgtttattaccacaaaaacaacaaaagttggatggaaagtgagttcagagtttattct
agtgcgaataattgcacttttgaatatgtctctcagccttttcttatggaccttgaaggaaaacagggtaatttcaaaaatcttagggaatttgtgtttaagaatat
tgatggttattttaaaatatattctaagcacacgcctattaatttagtgcgtgatctccctcagggtttttcggctttagaaccattggtagatttgccaataggtat
taacatcactaggtttcaaactttacttgctttacGtagaagttatttgactcctggtgattcttcttcaggttggacagctggtgctgcagcttattatgtgggtta
tcttcaacctaggacttttctattaaaatataatgaaaatggaaccattacagatgctgtagactgtgcacttgaccctctctcagaaacaaagtgtacgttgaa
atccttcactgtagaaaaaggaatctatcaaacttctaactttagagtccaaccaacagaatctattgttagatttcctaatattacaaacttgtgcccttttggtg
aagtttttaacgccaccagatttgcatctgtttatgcttggaacaggaagagaatcagcaactgtgttgctgattattctgtcctatataattccgcatcattttcc
acttttaagtgttatggagtgtctcctactaaattaaatgatctctgctttactaatgtctatgcagattcatttgtaattagaggtgatgaagtcagacaaatcgct
ccagggcaaactggaaagattgctgattataattataaattaccagatgattttacaggctgcgttatagcttggaattctaacaatcttgattctaaggttggtg
gtaattataattacctgtatagattgtttaggaagtctaatctcaaaccttttgagagagatatttcaactgaaatctatcaggccggtagcacaccttgtaatgg
tgttgaaggttttaattgttactttcctttacaatcatatggtttccaacccactaatggtgttggttaccaaccatacagagtagtagtactttcttttgaacttctac
atgcaccagcaactgtttgtggacctaaaaagtctactaatttggttaaaaacaaatgtgtcaatttcaacttcaatggtttaacaggcacaggtgttcttactg
agtctaacaaaaagtttctgcctttccaacaatttggcagagacattgctgacactactgatgctgtccgtgatccacagacacttgagattcttgacattaca
ccatgttcttttggtggtgtcagtgttataacaccaggaacaaatacttctaaccaggttgctgttctttatcaggatgttaactgcacagaagtccctgttgcta
ttcatgcagatcaacttactcctacttggcgtgtttattctacaggttctaatgtttttcaaacacgtgcaggctgtttaataggggctgaacatgtcaacaactc
atatgagtgtgacatacccattggtgcaggtatatgcgctagttatcagactcagcaatccatcattgcctacactatgtcacttggtgcagaaaattcagttg
cttactctaataactctattgccatacccacaaattttactattagtgttaccacagaaattctaccagtgtctatgaccaagacatcagtagattgtacaatgtac
atttgtggtgattcaactgaatgcagcaatcttttgttgcaatatggcagtttttgtacacaattaaaccgtgctttaactggaatagctgttgaacaagacaaaa
acacccaagaagtttttgcacaagtcaaacaaatttacaaaacaccaccaattaaagattttggtggttttaatttttcacaaatattaccagatccatcaaaac
caagcaagaggtcatttattgaagatctacttttcaacaaagtgacacttgcagatgctggcttcatcaaacaatatggtgattgccttggtgatattgctgcta
gagatctcatttgcgctcaaaaatttaacggacttacagttttaccacctttacttactgacgaaatgattgcgcaatatacatccgcattgttagccggaactat
tacatccggatggacttttggcgcaggcgTagcattacagattccattcgctatgcaaatggcttataggtttaacggtataggcgttacgcaaaacgtactt
tatgagaatcaaaaacttatcgctaaccaatttaattccgctatcggtaagattcaggattcattgtctagtactgctagtgcactcggtaagttgcaagacgta
gtgaatcaaaacgctcaagcacttaatacactcgttaaacagcttagttctaattttggcgcaatttctagtgtgcttaacgatatactatctagactcgataaag
tcgaagccgaagtgcaaatcgatagattgattaccggtaggttgcaatcattgcaaacatacgttacacagcaattgattagggccgcagagatacgcgct
agcgctaatctcgcagctactaaaatgtctgaatgcgtactcggacaatctaaacgtgtcgatttttgcggtaagggatatcatcttatgtcttttccacaatct
gcacctcacggagtcgtgtttttacacgttacttatgtgccagctcaagagaaaaattttacaaccgctcctgctatttgtcatgacggtaaggcacattttcct
agagagggcgtattcgtttctaacggtacacattggttcgttacacaacgtaatttttacgaacctcaaattattactactgataatacattcgtatcaggtaatt
gtgacgtagtgataggtatcgttaataatacagtttacgatccacttcaacctgaactcgatagttttaaagaggaactcgataagtattttaaaaatcatacat
cacctgacgtcgacttaggcgatatttcaggtattaacgctagtgtcgttaacattcaaaaagagattgatagacttaacgaagtcgctaaaaatcttaacga
atcacttatcgatctgcaagagttaggtaagtatgagcaatatattaaatggccttggtatatttggttaggctttatagccggattgatcgcaatcgttatggtt
acaattatgttatgttgtatgacatcatgttgttcatgtcttaagggatgttgttcatgcggatcatgttgtaaatttgacgaagacgattccgaaccagtgcttaa
aggcgttaagttacattatacataaacgaacttatggatttgtttatgagaatcttcacaattggaactgtaactttgaagcaaggtgaaatcaaggatgctact
ccttcagattttgttcgcgctactgcaacgataccgatacaagcctcactccctttcggatggcttattgttggcgttgcacttcttgctgtttttcagagcgcttc
caaaatcataaccctcaaaaagagatggcaactagcactctccaagggtgttcactttgtttgcaacttgctgttgttgtttgtaacagtttactcacaccttttg
ctcgttgctgctggccttgaagccccttttctctatctttatgctttagtctacttcttgcagagtataaactttgtaagaataataatgaggctttggctttgctgga
aatgccgttccaaaaacccattactttatgatgccaactattttctttgctggcatactaattgttacgactattgtataccttacaatagtgtaacttcttcaattgt
cattacttcaggtgatggcacaacaagtcctatttctgaacatgactaccagattggtggttatactgaaaaatgggaatctggagtaaaagactgtgttgtat
tacacagttacttcacttcagactattaccagctgtactcaactcaattgagtacagacactggtgttgaacatgttaccttcttcatctacaataaaattgttgat
gagcctgaagaacatgtccaaattcacacaatcgacggttcatccggagttgttaatccagtaatggaaccaatttatgatgaaccgacgacgactactag
cgtgcctttgtaagcacaagctgatgagtacgaacttatgtactcattcgtttcggaagagacaggtacgttaatagttaatagcgtacttctttttcttgctttcg
tggtattcttgctagttacactagccatccttactgcgcttcgattgtgtgcgtactgctgcaatattgttaacgtgagtcttgtaaaaccttctttttacgtttactct
cgtgttaaaaatctgaattcttctagagttcctgatcttctggtctaaacgaactaaatattatattagtttttctgtttggaactttaattttagccatggcagattcc
aacggtactattaccgttgaagagcttaaaaagctccttgaacaatggaacctagtaataggtttcctattccttacatggatttgtcttctacaatttgcctatgc
caacaggaataggtttttgtatataattaagttaattttcctctggctgttatggccagtaactttagcttgttttgtgcttgctgctgtttacagaataaattggatc
accggtggaattgctatcgcaatggcttgtcttgtaggcttgatgtggctcagctacttcattgcttctttcagactgtttgcgcgtacgcgttccatgtggtcat
tcaatccagaaactaacattcttctcaacgtgccactccatggcactattctgaccagaccgcttctagaaagtgaactcgtaatcggagctgtgatccttcgt
ggacatcttcgtattgctggacaccatctaggacgctgtgacatcaaggacctgcctaaagaaatcactgttgctacatcacgaacgctttcttattacaaatt
gggagcttcgcagcgtgtagcaggtgactcaggttttgctgcatacagtcgctacaggattggcaactataaattaaacacagaccattccagtagcagtg
acaatattgctttgcttgtacagtaagtgacaacagatgtttcatctcgttgactttcaggttactatagcagagatattactaattattatgaggacttttaaagttt
ccatttggaatcttgattacatcataaacctcataattaaaaatttatctaagtcactaactgagaataaatattctcaattagatgaagagcaaccaatggagat
tgattaaacgaacatgaaaattattcttttcttggcactgataacactcgctacttgtgagctttatcactaccaagagtgtgttagaggtacaacagtacttttaa
aagaaccttgctcttctggaacatacgagggcaattcaccatttcatcctctagctgataacaaatttgcactgacttgctttagcactcaatttgcttttgcttgt
cctgacggcgtaaaacacgtctatcagttacgtgccagatcagtttcacctaaactgttcatcagacaagaggaagttcaagaactttactctccaatttttctt
attgttgcggcaatagtgtttataacactttgcttcacactcaaaagaaagacagaatgattgaactttcattaattgacttctatttgtgctttttagcctttctgct
attccttgttttaattatgcttattatcttttggttctcacttgaactgcaagatcataatgaaacttgtcacgcctaaacgaacatgaaatttcttgttttcttaggaat
catcacaactgtagctgcatttcaccaagaatgtagtttacagtcatgtactcaacatcaaccatatgtagttgatgacccgtgtcctattcacttctattctaaat
ggtatattagagtaggagctagaaaatcagcacctttaattgaattgtgcgtggatgaggctggttctaaatcacccattcagtacatcgatatcggtaattat
acagtttcctgttcaccttttacaattaattgccaggaacctaaattgggtagtcttgtagtgcgttgttcgttctatgaagactttttagagtatcatgacgttcgt
gttgttttagatttcatctaaacgaacaaactaaaatgtctgataatggaccccaaaatcagcgaaatgcaccccgcattacgtttggtggaccctcagattca
actggcagtaaccagaatggagaacgcagtggggcgcgatcaaaacaacgtcggccccaaggtttacccaataatactgcgtcttggttcaccgctctc
actcaacatggcaaggaagaccttaaattccctcgaggacaaggcgttccaattaacaccaatagcagtccagatgaccaaattggctactaccgaagag
ctaccagacgaattcgtggtggtgacggtaaaatgaaagatctcagtccaagatggtatttctactacctaggaactgggccagaagctggacttccctat
ggtgctaacaaagacggcatcatatgggttgcaactgagggagccttgaatacaccaaaagatcacattggcacccgcaatcctgctaacaatgctgcaa
tcgtgctacaacttcctcaaggaacaacattgccaaaaggcttctacgcagaagggagcagaggcggcagtcaagcctcttctcgttcctcatcacgtagt
cgcaacagttcaagaaattcaactccaggcagcagtaggggaacttctcctgctagaatggctggcaatggcggtgatgctgctcttgctttgctgctgctt
gacagattgaaccagcttgagagcaaaatgtctggtaaaggccaacaacaacaaggccaaactgtcactaagaaatctgctgctgaggcttctaagaag
cctcggcaaaaacgtactgccactaaagcatacaatgtaacacaagctttcggcagacgtggtccagaacaaacccaaggaaattttggggaccaggaa
ctaatcagacaaggaactgattacaaacattggccgcaaattgcacaatttgcccccagcgcttcagcgttcttcggaatgtcgcgcattggcatggaagt
cacaccttcgggaacgtggttgacctacacaggtgccatcaaattggatgacaaagatccaaatttcaaagatcaagtcattttgctgaataagcatattgac
gcatacaaaacattcccaccaacagagcctaaaaaggacaaaaagaagaaggctgatgaaactcaagccttaccgcagagacagaagaaacagcaa
actgtgactcttcttcctgctgcagatttggatgatttctccaaacaattgcaacaatccatgagcagtgctgactcaactcaggcctaaactcatgcagacca
cacaaggcagatgggctatataaacgttttcgcttttccgtttacgatatatagtctactcttgtgcagaatgaattctcgtaactacatagcacaagtagatgta
gttaactttaatctcacatagcaatctttaatcagtgtgtaacattagggaggacttgaaagagccaccacattttcaccgaggccacgcggagtacgatcg
agtgtacagtgaacaatgctagggagagctgcctatatggaagagccctaatgtgtaaaattaattttagtagtgctatccccatgtgattttaatagcttctta
ggagaatgac

TABLE 2
Primers for Yellow Fever Virus

| | | SEQ | |
|---|---|---|---|
| | | ID | |
| Primer # | Primer Sequence | NO: | Primer Usage |
| 2519-YFVF1-F | AGCTTATCATCGATAAGCTTGCTAGC | 43 | for YFV Fragment 1 |
| | | | containing phi2.5 T7 |
| | | | promoter (1046 bp) |
| 2520-YFVF1-R | TGTCAGTAATTCCAATGCAGTGAG | 44 | for YFV Fragment 1 |
| | | | containing phi2.5 T7 |
| | | | promoter (1046 bp) |
| 2521-YFVF2-F | ATGACTGGAAGAATGGGTGAAAGG | 45 | for YFV Fragment 2 |
| | | | (1794 bp) |
| 2522-YFVF2-R | AGAGGCTTTCACTATTGATGCAAGC | 46 | for YFV Fragment 2 |
| | | | (1794 bp) |
| 2523-YFVF3-F | ATCAAGGATGCGCCATCAACTTTG | 47 | for YFV Fragment 3 |
| | | | (1550 bp) |
| 2524-YFVF3-R | AAGTCTCACCTCAGCCATAGTGAC | 48 | for YFV Fragment 3 |
| | | | (1550 bp) |
| 2525-YFVF4-F | AACGCCTTGTGCTGACCCTAG | 49 | for YFV Fragment 4 |
| | | | (1596 bp) |
| 2526-YFVF4-R | TTGGTTCCAACATCCTGTAAGTTAG | 50 | for YFV Fragment 4 |
| | | | (1596 bp) |
| 2527-YFVF5-F | ATCTTGGCCGAGTGCGCACG | 51 | for YFV Fragment 5 |
| | | | (1598 bp) |
| 2528-YFVF5-R | TCGGGGATCACAACCACCATC | 52 | for YFV Fragment 5 |
| | | | (1598 bp) |
| 2529-YFVF6-F | TGCTGTTTATACTGGCTGGACTAC | 53 | for YFV Fragment 6 |
| | | | (1601 bp) |
| 2530-YFVF6-R | TGGCATGTATGGAGCTAACACC | 54 | for YFV Fragment 6 |
| | | | (1601 bp) |
| 2531-YFVF7-F | ATCATCACCTTCAAGGACAAAACTG | 55 | for YFV Fragment 7 |
| | | | (1600 bp) |
| 2532-YFVF7-R | ATCCGTGCTCAGTGAGCCATG | 56 | for YFV Fragment 7 |
| | | | (1600 bp) |
| 2533-YFVF8-F | AGCCTACATGGATGTCATAAGTC | 57 | for YFV Fragment 8 |
| | | | (1460 bp) |
| 2534-YFVF8-R | AGTGGTTTTGTGTTTGTCATCCAAAG | 58 | for YFV Fragment 8 |
| | | | (1460 bp) |

In various embodiments, the viral RNA is from a wild-type Yellow fever virus, and the cDNA is cDNA encoding the viral RNA from the wild-type Yellow fever virus. In various embodiments, the viral RNA is from 17D Yellow fever virus, and the cDNA is cDNA encoding the viral RNA from the 17D Yellow fever virus. In various embodiments, the viral RNA is from 17D-204, 17DD, or 17D-213 Yellow fever virus, and the cDNA is cDNA encoding the viral RNA from the 17D-204, 17DD, or 17D-213 Yellow fever virus.

In various embodiments, each of the one or more overlapping cDNA fragments comprising the modified sequence comprises a sequence having one or more mutations relative to a corresponding sequence on the cDNA that results in one or more amino acid substitutions, additions or deletions. In certain embodiments, the one or more mutations relative to a corresponding sequence on the cDNA that results in 5 or more amino acid substitutions, additions or deletions. In certain embodiments, the one or more mutations relative to a corresponding sequence on the cDNA that results in 10 or more amino acid substitutions, additions or deletions. In certain embodiments, the one or more mutations relative to a corresponding sequence on the cDNA that results in 15 or more amino acid substitutions, additions or deletions. In certain embodiments, the one or more mutations relative to a corresponding sequence on the cDNA that results in 20 or more amino acid substitutions, additions or deletions. In certain embodiments, the one or more mutations relative to a corresponding sequence on the cDNA that results in 25 or more amino acid substitutions, additions or deletions.

In various embodiments, each of the one or more overlapping cDNA fragments comprising the modified sequence comprises a sequence encoding an amino acid sequence having up to 2% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence that results in having up to 1.75% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 1.5% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 1.25% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 1% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 0.75% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 0.5% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence that having up to 0.25% amino acid substitutions, additions or deletions relative to the amino acid sequence encoded by the corresponding sequence on the cDNA.

In particular embodiments, the method comprises performing RT-PCR on viral RNA from a wild-type RNA virus to generate cDNA (“wild-type cDNA”); performing PCR to generate and amplify 19 overlapping cDNA fragments from the wild-type cDNA, wherein the 19 overlapping cDNA fragments collectively encode the wild-type RNA virus; substituting an overlapping cDNA fragment comprising a deoptimized sequence for a corresponding overlapping cDNA fragment from the wild-type cDNA; performing overlapping and amplifying PCR to construct the modified viral genome comprising the deoptimized sequence.

In particular embodiments, the method comprises performing RT-PCR on viral RNA from a wild-type RNA virus to generate cDNA (“variant cDNA”); performing PCR to generate and amplify 19 overlapping cDNA fragments from the variant cDNA, wherein the 19 overlapping cDNA fragments collectively encode the variant RNA virus; substituting an overlapping cDNA fragment comprising a deoptimized sequence for a corresponding overlapping cDNA fragment from the variant cDNA; performing overlapping and amplifying PCR to construct the modified viral genome comprising the deoptimized sequence.

In various embodiments, the method comprises performing at least 1 passage of wild-type RNA viral isolate on permissive cells before performing the RT-PCR on the viral RNA from the RNA virus to generate the cDNA.

In various embodiments, the methods do not use an intermediate DNA clone, such as a plasmid, BAC or YAC. In various embodiments, the methods do not use a cloning host. In various embodiments, the methods do not include an artificial intron in the sequences; for example, to disrupt an offending sequence locus.

### Methods of Generating a Modified Infectious RNA

Various embodiments of the invention provide for a method of generating a modified infectious RNA, comprising: performing in vitro transcription of a modified viral genome to generate a modified RNA transcript.

In various embodiments, the method comprises generating the modified viral genome in accordance with embodiments of the present invention before performing the in vitro transcription.

Thus, in various embodiments, the method comprises performing reverse transcription polymerase chain reaction (“RT-PCR”) on a viral RNA from an RNA virus to generate cDNA; performing polymerase chain reaction (“PCR”) to generate and amplify two or more overlapping cDNA fragments from the cDNA, wherein the two or more overlapping cDNA fragments collectively encode the RNA virus; substituting one or more overlapping cDNA fragments comprising a modified sequence for one or more corresponding overlapping cDNA fragment generated from the viral RNA; performing overlapping and amplifying PCR to construct the modified viral genome, wherein the modified viral genome comprises one or more modified sequences; and performing in vitro transcription of a modified viral genome to generate a modified RNA transcript.

In other embodiments, the method comprises performing polymerase chain reaction (“PCR”) to generate and amplify two or more overlapping cDNA fragments from cDNA encoding viral RNA from an RNA virus, wherein the two or more overlapping cDNA fragments collectively encode the RNA virus, wherein one or more overlapping cDNA fragments comprises a modified sequence; performing overlapping and amplifying PCR to construct the modified viral genome, wherein the modified viral genome comprises one or more modified sequences; and performing in vitro transcription of a modified viral genome to generate a modified RNA transcript.

In other embodiments, the method comprises performing polymerase chain reaction (“PCR”) to generate and amplify two or more overlapping cDNA fragments from cDNA encoding viral RNA from an RNA virus, wherein the two or more overlapping cDNA fragments collectively encode the RNA virus; substituting one or more overlapping cDNA fragments comprising a modified sequence for one or more corresponding overlapping cDNA fragment generated from the viral RNA; performing overlapping and amplifying PCR to construct the modified viral genome, wherein the modified viral genome comprises one or more modified sequences; and performing in vitro transcription of a modified viral genome to generate a modified RNA transcript.

In various embodiments, the method further comprising extracting the viral RNA from the RNA virus prior to performing RT-PCR.

Additional embodiments of the modified viral genome and methods of generating the modified viral genome used in generating modified infectious RNA include the following: In various embodiments, performing overlapping PCR to construct the modified viral genome is done on the two or more overlapping cDNA fragments at the same time. Thus, if there are 5 more overlapping cDNA fragments, overlapping PCR to construct the modified viral genome is done on those 5 fragments at the same time. As further examples, if there are 8 more overlapping cDNA fragments, overlapping PCR to construct the modified viral genome is done on those 8 fragments at the same time; if there are 10 more overlapping cDNA fragments, overlapping PCR to construct the modified viral genome is done on those 10 fragments at the same time; if there are 15 more overlapping cDNA fragments, overlapping PCR to construct the modified viral genome is done on those 15 fragments at the same time; if there are 19 more overlapping cDNA fragments, overlapping PCR to construct the modified viral genome is done on those 19 fragments at the same time; if there are 20 more overlapping cDNA fragments, overlapping PCR to construct the modified viral genome is done on those 20 fragments at the same time; if there are 25 more overlapping cDNA fragments, overlapping PCR to construct the modified viral genome is done on those 25 fragments at the same time; and if there are 30 more overlapping cDNA fragments, overlapping PCR to construct the modified viral genome is done on those 30 fragments at the same time.

In various embodiments, the RNA virus is a negative strand RNA virus. Examples of negative strand RNA include those as are provided herein.

In other embodiments, the RNA virus is a positive strand RNA virus. Example of positive strand RNA include those as provided herein. Particular examples of positive strand RNA viruses include but are not limited coronavirus, including but not limited to Human coronavirus OC43, Human coronavirus HKU1, Middle East respiratory syndrome-related coronavirus (MERS-CoV), Severe acute respiratory syndrome coronavirus (SARS-CoV), and Severe acute respiratory syndrome coronavirus 2 (SARS-CoV-2) (including its variants). In various embodiments, the SARS-CoV-2 is the Alpha, Beta, Delta, or Gamma variant. Additional examples of positive strand RNA viruses include but are not limited to poliovirus, rhinovirus, hepatitis A virus, norovirus, Yellow fever virus, West Nile Virus, Hepatitis C virus, Dengue fever virus, Zika virus, and Rubella virus. In particular embodiments, the RNA virus is a Yellow fever virus. In yet particular embodiments, the RNA virus is 17D Yellow fever virus. In still other particular embodiments, the RNA virus is 17D-204, 17DD, or 17D-213.

In still other embodiments, the RNA virus is a double-stranded RNA virus. Examples of dsRNA viruses include those as provided herein.

In various embodiments, the virus is not Zika virus. In various embodiments, the virus is not Japanese encephalitis virus. In various embodiments, the virus is not West Nile virus. In various embodiments, the virus does not belong to the Flaviviridae family.

In various embodiments, each of the one or more overlapping cDNA fragments comprising the modified sequence comprises (1) a recoded sequence having reduced codon pair bias compared to a corresponding sequence on the cDNA, (2) at least 5 codons substituted with synonymous codons less frequently used, or (3) an increased number of CpG or UpA di-nucleotides compared to a corresponding sequence on the cDNA.

In embodiments wherein the modified sequence comprises a recoded sequence having reduced codon pair bias compared to a corresponding sequence on the cDNA, the recoded sequence has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the codon pair bias of the recoded sequence is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the corresponding sequence on the cDNA. In certain embodiments, it is in comparison corresponding sequence from which the calculation is to be made; for example, the corresponding sequence of a wild type virus.

In various embodiments, the corresponding sequence is at least 50 codons in length. In various embodiments, the corresponding sequence is at least 100 codons in length. In various embodiments, the corresponding sequence is at least 150 codons in length. In various embodiments, the corresponding sequence is at least 200 codons in length. In various embodiments, the corresponding sequence is at least 250 codons in length. In various embodiments, the corresponding sequence is at least 300 codons in length. In various embodiments, the corresponding sequence is at least 350 codons in length. In various embodiments, the corresponding sequence is at least 400 codons in length. In various embodiments, the corresponding sequence is at least 450 codons in length. In various embodiments, the corresponding sequence is at least 500 codons in length. In various embodiments, the corresponding sequence is the viral protein sequence. In various embodiments, the corresponding sequence is the sequence of the entire virus.

In various embodiments, “similar amino acid sequence” as used herein refers to an amino acid sequence having less than 2% amino acid substitutions, deletions or additions compared to the comparison sequence. In various embodiments, if specifically provided for in the claims, “similar amino acid sequence” refers to an amino acid sequence having less than 1.75% amino acid substitutions, deletions or additions compared to the comparison sequence. In various embodiments, if specifically provided for in the claims, “similar amino acid sequence” refers to an amino acid sequence having less than 1.5% amino acid substitutions, deletions or additions compared to the comparison sequence. In various embodiments, if specifically provided for in the claims, “similar amino acid sequence” refers to an amino acid sequence having less than 1.25% amino acid substitutions, deletions or additions compared to the comparison sequence. In various embodiments, if specifically provided for in the claims, “similar amino acid sequence” refers to an amino acid sequence having less than 1% amino acid substitutions, deletions or additions compared to the comparison sequence. In various embodiments, if specifically provided for in the claims, “similar amino acid sequence” refers to an amino acid sequence having less than 0.75% amino acid substitutions, deletions or additions compared to the comparison sequence. In various embodiments, if specifically provided for in the claims, “similar amino acid sequence” refers to an amino acid sequence having less than 0.5% amino acid substitutions, deletions or additions compared to the comparison sequence. In various embodiments, if specifically provided for in the claims, “similar amino acid sequence” refers to an amino acid sequence having less than 0.25% amino acid substitutions, deletions or additions compared to the comparison sequence.

In various embodiments, an amino acid sequence having a deletion of a furin cleavage site in considered a similar amino acid sequence. For example, for SARS-CoV-2, a 36 nt deletion is in the Spike gene (genome position 23594-23629). The deletion encompasses the 12 amino acids TNSPRRARSVAS (SEQ ID NO:2) that include the polybasic furin cleavage site. The furin cleavage site in SARS-CoV2 Spike has been proposed as a potential driver of the highly pathogenic phenotype of SARS-CoV2 in the human host. While not wishing to be bound by any particular theory, we believe that absence of the furin cleavage is beneficial to the SARS-CoV-2 virus growth in vitro in Vero cells, and that the deletion evolved during passaging in Vero cell culture. We further believe that the absence of the furin cleavage site may contribute to attenuation in the human host of a SARS-CoV-2 virus carrying such mutation.

In embodiments wherein the modified sequence comprises at least 5 codons substituted with synonymous codons less frequently used, the modified sequence comprises at least 10, or at least 30, or at least 30, or at least 40, or at least 50, or at least 75, or at least 100, at least 150, or at least 200, or at least 250 substituted with synonymous codons less frequently used. In certain embodiments, the modified sequence comprises at least 20 codons substituted with synonymous codons less frequently used. In certain embodiments, the modified sequence comprises at least 50 codons substituted with synonymous codons less frequently used.

In some embodiments, the substitution of synonymous codons is with those that are less frequent in the viral host; for example, human. Other examples of viral hosts include but are not limited to those noted above. In some embodiments, the substitution of synonymous codons is with those that are less frequent in the virus itself.

In embodiments wherein the modified sequence comprises an increased number of CpG or UpA di-nucleotides compared to a corresponding sequence (for example, on the cDNA), the increase is of about 15-55 CpG or UpA di-nucleotides compared the corresponding sequence. In various embodiments, increase is of about 15, 20, 25, 30, 35, 40, 45, or 55 CpG or UpA di-nucleotides compared the corresponding sequence. In some embodiments, the increased number of CpG or UpA di-nucleotides compared to a corresponding sequence (e.g., on the cDNA) is about 10-75, 15-25, 25-50, or 50-75 CpG or UpA di-nucleotides compared the corresponding sequence.

In various embodiments, performing PCR to generate and amplify two or more overlapping cDNA fragments from the cDNA comprises using two or more primer pairs, each pair specific for each of the overlapping cDNA fragments. In various embodiments, performing PCR to generate and amplify two or more overlapping cDNA fragments from the cDNA comprises using two or more primer pairs selected from Table 1. In various embodiments, performing PCR to generate and amplify two or more overlapping cDNA fragments from the cDNA comprises using two or more primer pairs selected from Table 2.

In various embodiments, the length of the primers is about 15-55 base pairs (bp) in length. In various embodiments, the length of the primers is about 19-55 bp in length. In various embodiments, the length of the primers is about 10-65 bp in length. In various embodiments, the length of the primers is about 16-20, 21-25, 26-30, 31-35, 36-40, 41-45, 46-50, 51-55, 56-60, or 61-65 bp in length.

In various embodiments, performing PCR to generate and amplify two or more overlapping cDNA fragments from the cDNA comprises using 5 or more primer pairs, each pair specific for each of the overlapping cDNA fragments. In various embodiments, the two or more overlapping cDNA fragments from the cDNA is 5 or more overlapping cDNA fragments and the 5 or more overlapping cDNA fragments collectively encode the RNA virus. In various embodiments, performing PCR to generate and amplify 5 or more overlapping cDNA fragments from the cDNA comprises using 5 or more primer pairs selected from Table 1. In various embodiments, performing PCR to generate and amplify 5 or more overlapping cDNA fragments from the cDNA comprises using 5 or more primer pairs selected from Table 2.

In various embodiments, performing PCR to generate and amplify two or more overlapping cDNA fragments from the cDNA comprises using 8 or more primer pairs, each pair specific for each of the overlapping cDNA fragments. In various embodiments, the two or more overlapping cDNA fragments from the cDNA is 8 or more overlapping cDNA fragments and the 8 or more overlapping cDNA fragments collectively encode the RNA virus. In various embodiments, performing PCR to generate and amplify 8 or more overlapping cDNA fragments from the cDNA comprises using 8 or more primer pairs selected from Table 1. In various embodiments, performing PCR to generate and amplify 8 or more overlapping cDNA fragments from the cDNA comprises using 8 or more primer pairs selected from Table 2.

In various embodiments, performing PCR to generate and amplify two or more overlapping cDNA fragments from the cDNA comprises using 10 or more primer pairs, each pair specific for each of the overlapping cDNA fragments. In various embodiments, the two or more overlapping cDNA fragments from the cDNA is 10 or more overlapping cDNA fragments and the 10 or more overlapping cDNA fragments collectively encode the RNA virus. In various embodiments, performing PCR to generate and amplify 10 or more overlapping cDNA fragments from the cDNA comprises using 10 or more primer pairs selected from Table 1.

In various embodiments, performing PCR to generate and amplify two or more overlapping cDNA fragments from the cDNA comprises using 15 or more primer pairs, each pair specific for each of the overlapping cDNA fragments. In various embodiments, the two or more overlapping cDNA fragments from the cDNA is 15 or more overlapping cDNA fragments and the 15 or more overlapping cDNA fragments collectively encode the RNA virus. In various embodiments, performing PCR to generate and amplify 15 or more overlapping cDNA fragments from the cDNA comprises using 15 or more primer pairs selected from Table 1.

In various embodiments, the two or more overlapping cDNA fragments from the cDNA is 20 or more overlapping cDNA fragments and the 20 or more overlapping cDNA fragments collectively encode the RNA virus. In various embodiments, performing PCR to generate and amplify 20 or more overlapping cDNA fragments from the cDNA comprises using 20 or more primer pairs, each pair specific for each overlapping cDNA fragments.

In various embodiments, the two or more overlapping cDNA fragments from the cDNA is 25 or more overlapping cDNA fragments and the 25 or more overlapping cDNA fragments collectively encode the RNA virus. In various embodiments, performing PCR to generate and amplify 25 or more overlapping cDNA fragments from the cDNA comprises using 25 or more primer pairs, each pair specific for each overlapping cDNA fragments.

In various embodiments, the two or more overlapping cDNA fragments from the cDNA is 30 or more overlapping cDNA fragments and the 30 or more overlapping cDNA fragments collectively encode the RNA virus. In various embodiments, performing PCR to generate and amplify 30 or more overlapping cDNA fragments from the cDNA comprises using 30 or more primer pairs, each pair specific for each overlapping cDNA fragments.

In various embodiments, the two or more overlapping cDNA fragments from the cDNA is 19 overlapping cDNA fragments and the 19 overlapping cDNA fragments collectively encode the RNA virus; for example, the SARS-CoV-2 or SARS-CoV-2 variant (e.g., Alpha, Beta, Delta, or Gamma). In various embodiments, performing PCR to generate and amplify 19 overlapping cDNA fragments from the first cDNA comprises using all 19 primer pairs from Table 1.

In various embodiments, the two or more overlapping cDNA fragments from the cDNA is 8 overlapping cDNA fragments and the 8 overlapping cDNA fragments collectively encode the RNA virus, for example, the Yellow Fever Virus (e.g., 17D, 17DD, 17D-213, 17D-204). In various embodiments, performing PCR to generate and amplify 8 overlapping cDNA fragments from the first cDNA comprises using all 8 primer pairs from Table 2.

In various embodiments, the two or more overlapping cDNA fragments is 2-30 fragments. In various embodiments, the two or more overlapping cDNA fragments is 2-5 fragments. In various embodiments, the two or more overlapping cDNA fragments is 6-8 fragments. In various embodiments, the two or more overlapping cDNA fragments is 8-10 fragments. In various embodiments, the two or more overlapping cDNA fragments is 11-15 fragments. In various embodiments, the two or more overlapping cDNA fragments is 16-20 fragments. In various embodiments, the two or more overlapping cDNA fragments is 21-25 fragments. In various embodiments, the two or more overlapping cDNA fragments is 26-30 fragments.

In various embodiments, the length of the overlap is about 40-400 bp. In various embodiments, the length of the overlap is about 200 bp. In various embodiments, the length of the overlap is about 40-100 bp. In various embodiments, the length of the overlap is about 100-200 bp. In various embodiments, the length of the overlap is about 100-150 bp. In various embodiments, the length of the overlap is about 150-200 bp. In various embodiments, the length of the overlap is about 200-250 bp. In various embodiments, the length of the overlap is about 200-300 bp. In various embodiments, the length of the overlap is about 300-400 bp.

In various embodiments, the viral RNA is from a wild-type RNA virus, and the cDNA is cDNA encoding the viral RNA from the wild-type RNA virus (“wild-type cDNA”).

In various embodiments, the viral RNA is from a wild-type SARS-CoV-2, and the cDNA is cDNA encoding the viral RNA from the wild-type SARS-CoV-2. In various embodiments, the viral RNA is from a variant SARS-CoV-2, and the cDNA is cDNA encoding the viral RNA from the variant SARS-CoV-2. In various embodiments, the variant is the Alpha variant, Beta variant, Delta variant, or Gamma variant.

In various embodiments, the viral RNA is from a wild-type Yellow fever virus, and the cDNA is cDNA encoding the viral RNA from the wild-type Yellow fever virus. In various embodiments, the viral RNA is from 17D Yellow fever virus, and the cDNA is cDNA encoding the viral RNA from the 17D Yellow fever virus. In various embodiments, the viral RNA is from 17D-204, 17DD, or 17D-213 Yellow fever virus, and the cDNA is cDNA encoding the viral RNA from the 17D-204, 17DD, or 17D-213 Yellow fever virus.

In various embodiments, each of the one or more overlapping cDNA fragments comprising the modified sequence comprises a sequence having one or more mutations relative to a corresponding sequence on the cDNA that results in one or more amino acid substitutions, additions or deletions. In certain embodiments, the one or more mutations relative to a corresponding sequence on the cDNA that results in 5 or more amino acid substitutions, additions or deletions. In certain embodiments, the one or more mutations relative to a corresponding sequence on the cDNA that results in 10 or more amino acid substitutions, additions or deletions. In certain embodiments, the one or more mutations relative to a corresponding sequence on the cDNA that results in 15 or more amino acid substitutions, additions or deletions. In certain embodiments, the one or more mutations relative to a corresponding sequence on the cDNA that results in 20 or more amino acid substitutions, additions or deletions. In certain embodiments, the one or more mutations relative to a corresponding sequence on the cDNA that results in 25 or more amino acid substitutions, additions or deletions.

In various embodiments, each of the one or more overlapping cDNA fragments comprising the modified sequence comprises a sequence encoding an amino acid sequence having up to 2% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence that results in having up to 1.75% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 1.5% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 1.25% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 1% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 0.75% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence having up to 0.5% amino acid substitutions, additions or deletions relative to the amino acid sequence encoded by the corresponding sequence on the cDNA. In various embodiments, each of the one or more overlapping cDNA fragments comprising the modified sequence comprises a sequence encoding an amino acid sequence that having up to 0.25% amino acid substitutions, additions or deletions relative to the amino acid sequence encoded by the corresponding sequence on the cDNA.

In particular embodiments, the method comprises performing RT-PCR on viral RNA from a wild-type RNA virus to generate cDNA (“wild-type cDNA”); performing PCR to generate and amplify 19 overlapping cDNA fragments from the wild-type cDNA, wherein the 19 overlapping cDNA fragments collectively encode the wild-type RNA virus; substituting an overlapping cDNA fragment comprising a deoptimized sequence for a corresponding overlapping cDNA fragment from the wild-type cDNA; performing overlapping and amplifying PCR to construct the modified viral genome comprising the deoptimized sequence.

In particular embodiments, the method comprises performing RT-PCR on viral RNA from a wild-type RNA virus to generate cDNA (“variant cDNA”); performing PCR to generate and amplify 19 overlapping cDNA fragments from the variant cDNA, wherein the 19 overlapping cDNA fragments collectively encode the variant RNA virus; substituting an overlapping cDNA fragment comprising a deoptimized sequence for a corresponding overlapping cDNA fragment from the variant cDNA; performing overlapping and amplifying PCR to construct the modified viral genome comprising the deoptimized sequence.

In various embodiments, the methods do not use an intermediate DNA clone such as a plasmid, BAC or YAC. In various embodiments, the methods do not use a cloning host. In various embodiments, the methods do not include an artificial intron in the sequences; for example, to disrupt offending sequence locus.

Additional embodiments of the modified viral genome and methods of generating the modified viral genome are as provided herein and are included in these embodiments of generating the modified infectious RNA.

### Methods of Generating a Modified Virus

Various embodiments of the invention provide for a method of generating a modified virus, comprising transfecting host cells with a quantity of a modified infectious RNA; culturing the host cells; and collecting infection medium comprising the modified virus.

In various embodiments, the method further comprises generating the quantity of modified infectious RNA in accordance with various embodiments of the present invention before transfecting host cells with the quantity of the modified infectious RNA. Thus, the invention comprises performing in vitro transcription of a modified viral genome to generate a modified RNA transcript; and transfecting host cells with a quantity of a modified infectious RNA; culturing the host cells; and collecting infection medium comprising the modified virus.

In other embodiments, the method comprises performing reverse transcription polymerase chain reaction (“RT-PCR”) on a viral RNA from an RNA virus to generate cDNA; performing polymerase chain reaction (“PCR”) to generate and amplify two or more overlapping cDNA fragments from the cDNA, wherein the two or more overlapping cDNA fragments collectively encode the RNA virus; substituting one or more overlapping cDNA fragments comprising a modified sequence for one or more corresponding overlapping cDNA fragment generated from the viral RNA; performing overlapping and amplifying PCR to construct the modified viral genome, wherein the modified viral genome comprises one or more modified sequences; performing in vitro transcription of a modified viral genome to generate a modified RNA transcript; transfecting host cells with a quantity of a modified infectious RNA; culturing the host cells; and collecting infection medium comprising the modified virus.

In other embodiments, the method comprises performing polymerase chain reaction (“PCR”) to generate and amplify two or more overlapping cDNA fragments from cDNA encoding viral RNA from an RNA virus, wherein the two or more overlapping cDNA fragments collectively encode the RNA virus, wherein one or more overlapping cDNA fragments comprises a modified sequence; performing overlapping and amplifying PCR to construct the modified viral genome, wherein the modified viral genome comprises one or more modified sequences; performing in vitro transcription of a modified viral genome to generate a modified RNA transcript; and transfecting host cells with a quantity of a modified infectious RNA; culturing the host cells; and collecting infection medium comprising the modified virus.

In other embodiments, the method comprises performing polymerase chain reaction (“PCR”) to generate and amplify two or more overlapping cDNA fragments from cDNA encoding viral RNA from an RNA virus, wherein the two or more overlapping cDNA fragments collectively encode the RNA virus; substituting one or more overlapping cDNA fragments comprising a modified sequence for one or more corresponding overlapping cDNA fragment generated from the viral RNA; performing overlapping and amplifying PCR to construct the modified viral genome, wherein the modified viral genome comprises one or more modified sequences; performing in vitro transcription of a modified viral genome to generate a modified RNA transcript; and transfecting host cells with a quantity of a modified infectious RNA; culturing the host cells; and collecting infection medium comprising the modified virus.

In various embodiments, the method further comprising extracting the viral RNA from the RNA virus prior to performing RT-PCR.

In various embodiments, the methods do not use an intermediate DNA clone such as a plasmid, BAC or YAC. In various embodiments, the methods do not use a cloning host. In various embodiments, the methods do not include an artificial intron in the sequences; for example, to disrupt offending sequence locus.

Specific embodiments of the modified viral genome, methods of generating the modified viral genome, and the infectious RNA and generating the infectious RNA are as provided above and below and are included in these embodiments of generating these modified viruses.

Example of host cells include, but are not limited to Vero E6 cells, MDCK cells, HeLa cells, Chicken embryo fibroblasts, embryonated chicken eggs, MRC-5 cells, WISTAR cells, PERC.6 cells, Huh-7 cells, BHK cells, MA-104 cells, Vero cells, WI-38 cells, and HEK 293 cells.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Procedures

### RT-PCR

Coronavirus strain 2019-nCoV/USA-WA1/2020 (“WA1”) (BEI Resources NR-52281, Lot 70034262) was distributed by BEI Resources after 3 passages on Vero (CCL81) at CDC, and one passage on Vero E6 at BEI Resources. The full virus genome sequence after 4 passages was determined by CDC and found to contain no nucleotide differences (Harcourt et al., 2020) compared to the clinical specimen from which it was derived (Genbank Accession MN985325) Upon receipt, WA1 was amplified by a further two passages on Vero E6 cells in DMEM containing 2% FBS at 3TC.

Passage 6 WA1 virus was used to purify viral genome RNA by extraction with Trizol reagent (Thermo Fisher) according to standard protocols. Briefly, 0.5 ml virus sample with a titer of 1×10\hat{ }7 PFU/ml was extracted with an equal volume of Trizol. The procedure had previously been validated in four separate experiment to completely inactivate SARS-CoV2 virus infectivity. After phase separation by addition of 0.1 ml chloroform, the RNA in aqueous phase was precipitated with an equal volume of isopropanol. The precipitated RNA was washed in 70% ethanol, dried, and resuspended in 20 ul RNAse-free water.

Viral cDNA Generation

Wild-type cDNA were synthesized using SuperScript IV First Strand Synthesis system. In each reaction, a total reaction volume of 13 μl for Tube #1 was set up as follows:
1. 50 μM Oligo d(T)20: 1 ul (Alternatively, primer #1822 (10 μM): 1 μl)
2. 50 ng/μl Random Hexamer: 1 μl
3. 10 mM dNTP: 1 μl
4. WT RNA: 2-10 μl
5. H₂O: add to 13 μl

The sample was mixed and incubated at 65° C. for 5 minutes, then immediately put on ice for 1 minute. Another tube (Tube #2) was prepared with a total reaction volume of 7 μl:
1. 5× Buffer: 4 μl
2. 100 mM DTT:
3. Rnase Inhibitor (40 U/μl): 1 μl (optional)
4. SuperScipt IV enzyme: 1 μl

We mixed Tube #1 and Tube #2, for a total reaction volume of 20 μl, and incubated at 23° C. for 10 minutes, followed by 50° C. for 50 minutes, and 80° C. for 10 minutes to generate cDNA.

### Overlapping Polymerase Chain Reaction

Q5 High-Fidelity 2× Master Mixture (NEB, Ipswich, Massachusetts) were used to amplify genome fragments from cDNA.

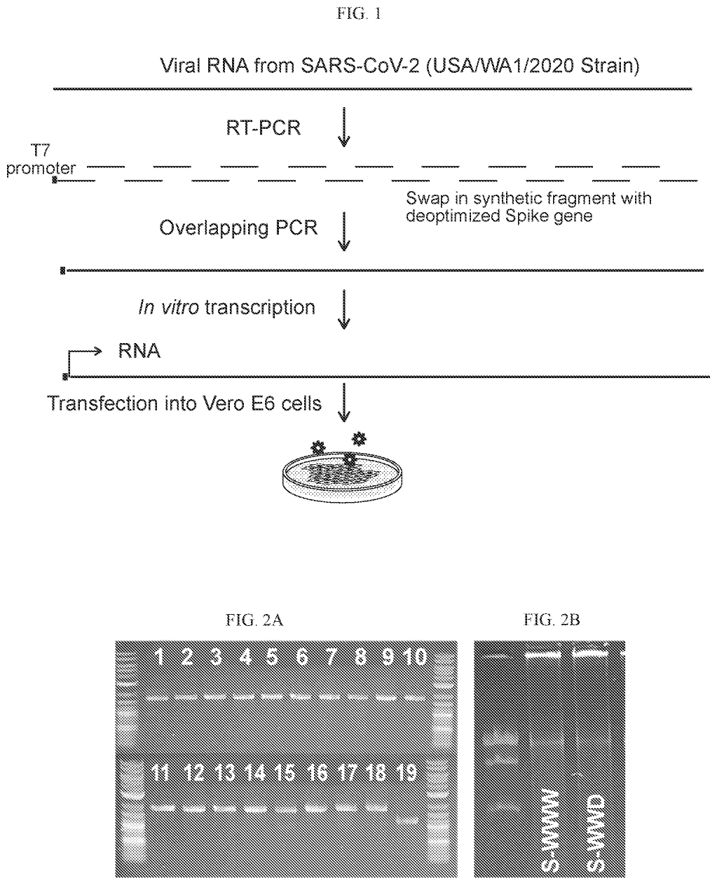

The 20 μl reaction containing 1 μl fresh-made cDNA, 1 μl of forward and reverse primers (detailed in Table 1) at 0.5 μM concentration, 10 μl of the 2×Q5 master mixture and H₂O. Reaction parameters were as follows: 98° C. 30 sec to initiate the reaction, followed by 30 cycles of 98° C. for 10 sec, 60° C. for 30 seconds or 45 seconds, and 65° C. for 1 min and a final extension at 65° C. for 5 min. Totally 19 genome fragments, all about 1.8 Kb except fragment 19 (about 1.2 Kb) were obtained, which cover the whole viral genome with 200 bp overlapping region between any two of them using specific primers (Table 1). Amplicons were verified by agarose gel electrophoresis (FIG. 2A) and purified using the QIAquick PCR Purification Kit (Qiagen). Elutions were quantified by Nanodrop.

Q5® High-Fidelity DNA Polymerase (NEB, Ipswich, Massachusetts) were used to reconstruct the whole COVID-19 genome.

First, all 19 genome fragments were used in an overlapping reaction to reconstruct the full genome. Briefly, a mixture with 30-40 ng of each DNA fragment (the molar ratio among all pieces are at 1:1), 10 μl 5× reaction buffer, 1 μl 10 mM dNTP, 0.5 μl Q5 polymerase and H₂O to a final volume of 50 μl was made. The reaction was carried out under following condition: 98° C. for 30 sec, and 72° C. for 16 min 30 sec for 10 cycles.

Next, 41 overlapping reaction product were mixed with 4 μl 5× reaction buffer, 1 μl 10 mM dNTP, 1 μl of each flanking primers at 0.5 μM, 0.41 Q5 polymerase and H₂O to a final volume of 20 μl and PCR was carried out as follows: 98° C. 30 sec to initiate the reaction, followed by 15 cycles of 98° C. for 10 sec, 60° C. for 45 sec, and 72° C. for 16 minutes 30 seconds, and a final extension at 65° C. for 5 min. To check the results, 5 μl PCR product was visualized on 0.4% agarose gel (FIG. 2B).

### In Vitro Transcription

DNA templates amplified from full-length PCR were purified using conventional phenol/chloroform extraction followed by Ethanol precipitation in the presence of 3M Sodium Acetate prior to RNA work. RNA transcripts was in vitro synthesized using the HiScribe T7 Transcription Kit (New England Biolabs) according to the manufacturer's instruction with some modifications. A 20 μl reaction was set up by adding 500 ng DNA template and 2.4 μl 50 mM GTP (cap analog-to-GTP ratio is 1:1). The reaction was incubated at 37° C. for 3 hr. Then RNA was precipitated and purified by Lithium Chloride precipitation and washed once with 70% Ethanol. The N gene DNA template was also prepared by PCR from cDNA using specific forward primer (2320-N-F: GAAtaatacgactcactataggGACGTTCGTGTTGTTTTAGATTTCATCTAAACG (SEQ ID NO:41), the lowercase sequence represents T7 promoter; the underlined sequence represents the 5′ NTR upstream of the N gene ORF) and reverse primer (2130-N-R, tttttttttttttttttttttGTCATTCTCCTAAGAAGCTATTAAAATCACATGG (SEQ ID NO:42)).

### Transfection of Vero E6 Cells by RNA Electroporation

Vero E6 cells were obtained from ATCC (CRL-1586) and maintained in DMEM high glucose supplemented with 10% FBS. To transfect viral RNA, 10 μs of purified full length genome RNA transcripts, together with 5 ug of capped WA1-N mRNA, were electroporated into Vero E6 cells using the Maxcyte ATX system according manufacturer's instructions. Briefly, 3-4×10⁶ Vero E6 cells were once washed in Maxcyte electroporation buffer and resuspended in 100 μl of the same. The cell suspension was mixed gently with the RNA sample, and the RNA/cell mixture transferred to Maxcyte OC-100 processing assemblies. Electroporation was performed using the pre-programmed Vero cell electroporation protocol. After 30 minutes recovery of the transfected cells at 37 C/5% CO₂, cells were resuspended in warm DMEM/10% FBS and distributed among three T25 flasks at various seeding densities (1/2, 1/3, 1/6 of the total cells). Transfected cells were incubated at 37° C./5% CO₂ for 6 days or until CPE appeared. Infection medium was collected on days 2, 4, and 6, with completely media change at day 2 and day 4 (DMEM/5% FBS). The generated viruses were detectable by plaque assay as early as 2 days post transfection, with peak virus generation between days 4-6.

### Passaging of Stock Virus and Plaque Titration of SARS-CoV-2 in Vero E6 Cells

Serial 10-fold dilutions were prepared in DMEM/2% FBS. 0.5 ml of each dilution were added to 12-wells of Vero E6 cells that were 80% confluent. After 1 hour incubation at 3TC, the inoculum was removed, and 2 ml of semisolid overlay was added per well, containing 1×DMEM, 0.3% Gum Tragacanth, 2% FBS and 1× Penicillin/Streptomycin. After 3 or 4 day incubation at 37° C./5% CO₂ the overlay was removed, wells were rinsed gently with PBS, followed by fixation and staining with Crystal Violet.

### Results

Generation of individual genome fragments 1-19 and the whole genomic DNA generated by overlapping PCR went well, with clear bands visible on 0.4% agarose gels (FIG. 2A).

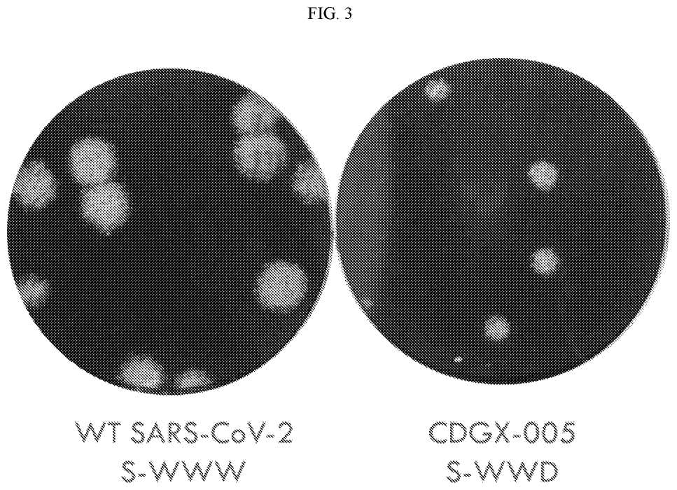

In vitro transcription produced RNA used to transfect Vero E6 cells with S-WWW (WT) and S-WWD and recover live virus that was titrated in Vero E6 cells. After incubation for 3 days, the plaque assays were stained and we observed smaller plaques observed in the partially spike-deoptimized 5-WWD candidate (FIG. 3) and a 40% reduced final titer.

### Example 2

An exemplary CDX-005 construct design is shown in FIG. 1. The CDX-005 pre-master virus seed (preMVS) was developed as follows: RNA of SARS-COV-2 BetaCoV/USA/WA1/2020 was extracted from infected, characterized Vero E6 cells (ATCC CRL-1586 Lot #70010177) and converted to 19 overlapping DNA fragments by RT-PCR using commercially available reagents and kits. Overlapping PCR was used to stitch together 19 1.8 kb wt genome fragments along with one deoptimized Spike gene cassette. Specifically, 1,272 nucleotides of the Spike ORF were human codon pair deoptimized from genome position 24115-25387 resulting in 283 silent mutations changes relative to parental WA1/2020 virus. The resulting full-length cDNA was transcribed in vitro to make full-length viral RNA. Viral recovery was conducted in a new BSL-3 laboratory at Stony Brook University (NY) that was commissioned for the first time in April 2020, with our project being the only project ever to occur in the lab. This viral RNA was then electroporated in characterized Vero E6 cells (Lot #70010177). This yielded CDX-005 virus (FIG. 3) that was subsequently passaged an additional time on Vero E6 cells to yield passage 1, P1 (Lot #1-060820-9-1). P1 material was used in the hamster study described below.

### Example 3

### Synthesis of SARS-CoV-2 Alpha Variant, Beta Variant and Delta Variant

Synthesis of the Alpha variant, Beta variant and the Delta Variant is similar as described for the deoptimized SARS-CoV-2, Coronavirus strain 2019-nCoV/USA-WA 1/2020 described above, with exception that the fragments carrying the mutations of each variant were used.

Key mutations for each variant within the Spike gene were identified. About 6-10 sequences of the variant were selected from GISAID and a multi-alignment using BLASTn comparing to our original WT design or CDX-005 (with deoptimization in Spike).

Once the nucleotide mutations were identified, the codons of the Deoptimized Coronavirus strain 2019-nCoV/USA-WA1/2020 design (noted above) were replaced with the codons from the variants. If the mutation resulted in a deletion, the same deletion was made for the deoptimized sequence of the variant.

Thereafter, the DNA fragments carrying these mutations were synthesized. The Spike gene was separated into 3 fragments, herein referred to as F14, F15, and F16. F16 contained the deoptimized regions. Based on the location of the mutations, either 2 or all 3 of these fragments were synthesized.

Briefly, after all 19 fragments were obtained by PCR/RT-PCR process, overlapping PCR was performed to construct the viral genome, followed by in vitro transcription and Vero E6 transfection. The same primers were used as described above for CDX-005.

### Example 4

### Synthesis of Deoptimized Yellow Fever Virus

Codon pair deoptimized cassettes are introduced into the 17D viral genome by reverse genetics methods to “over-attenuate” the resulting virus. The over-attenuation provides a safety “buffer” that will allow to absorb potential de-attenuating effects of mutations that may occur upon virus adaptation when switching the manufacturing substrate of the vaccine from chick embryos to cell culture.

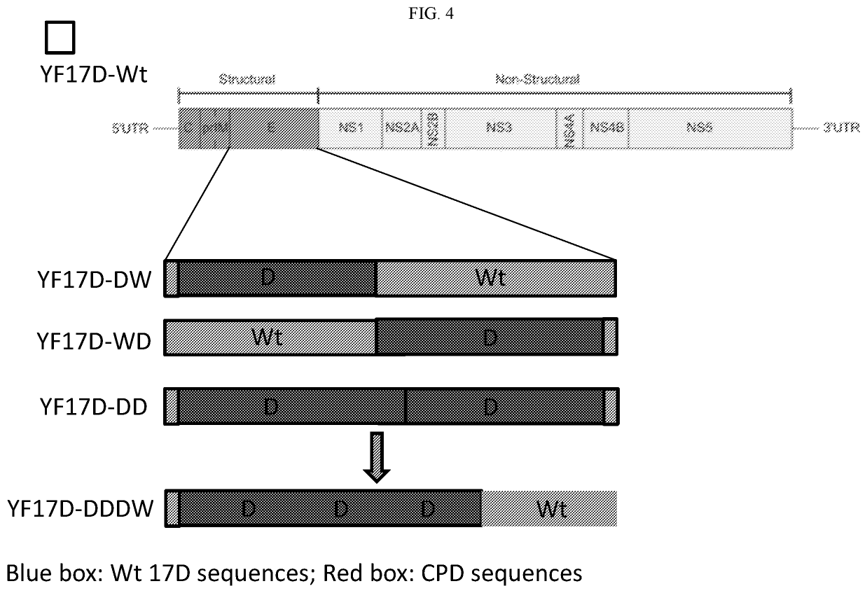

The published full length Yellow Fever Virus Vaccine (17D) genome sequence (Genbank Accession #JN628279, as of Jun. 28, 2021, herein incorporated by reference) was divided in silico into 8 fragments with overlapping region at both ends. Fragments 1 and 3-8 correspond to the backbone 17D genome and are constant in the virus designs describe in this example. Fragment 2, encoding the E glycoprotein was deoptimized. See FIG. 4. Four versions of Fragment 2 (all encoding same amino acid sequence) were initially synthesized. F2-WW represents the sequence of the YF vaccine strain 17D. A synthetic 17D virus carrying the F2-WW cassette corresponds to a cloned version of the current 17D vaccine strain. In F2-DW, and F2-WD, either the first half or the second half of the E-glycoprotein are deoptimized, respectively. Introduction of F2-DW, and F2-WD into the 17D genome produces vaccine candidates YF-DW and YF-WD, respectively. F2-DD contains a wholly deoptimized E-glycoprotein, and the resulting YF-DD virus is expected to be the most highly attenuated vaccine candidate of the four viruses (YF-WW, YF-DW, YF-WD, YF-DD) currently contemplated. The recovery YF-DD is described herein. However, the recovery method is applicable to YF-WW, YF-DW, YF-WD, and other YF deoptimized virus candidates.

The seven backbone fragments F1, F3-8, and four variations of F2 were synthesized de novo (BioBasic, Markham Ontario) and delivered as sequence confirmed plasmids (in low copy number vector pBR322).

Upon receiving synthetic plasmids from BioBasic, all fragments were PCR amplified and purified. Full length overlapping PCR were performed to obtain full length YF-DD DNA genome flanked by 3′ T7 RNA polymerase promoter. T7 in vitro transcription was used to generate infectious full length YF-DD genome RNA genome, which was used to recover YF-DD virus by transfection in animal origin free Vero (WHO 10-87) cells.

The above procedures were repeated with an additional version of F2. F2-DDDW contains a longer deoptimized region, wherein approximately the first 314th of the E-glycoprotein is deoptimized, as shown in FIG. 4.

### Experimental Procedures:

Cells—Vero WHO 10-87 (MCB+19 passages); animal origin free culture

Medium and reagents used: OptiPRO SFM, DMEM, NEB Q5, DPBS, mMESSAGE mMACHINE™ T7 Transcription Kit, Lipofectamine™ MessengerMAX™ Transfection Reagent PCR for Each Fragment

NEB Q5 polymerase was used to amplify all 8 genome fragments, synthesized by BioBasics, as building blocks for downstream overlapping PCR. ing of each plasmids works as templates, amplified with gene specific primers (0.2 uM) in a 40 ul system. All PCR products were purified by DNAland Gel Extraction PCR Purification 2-in-1 Kit.

### Overlapping PCR for Full Length YF-DD

After purifying each PCR products, a mix of 0.02 pmol of each DNA fragment were used to generate full length YF-DD by overlapping PCR. Reaction volume was kept as 20 ul. Conditions were: 98° C. for 30 sec, and 72° C. for 4 min 30 sec for 10 cycles. No primers were used at this step.

After the initial step, 2 ul of overlapping PCR product were mixed with 0.1 uM Forward primer #2519 and Reverse primer #2534, as well as 2×Q5 to amplify the full length YF-DD. Reaction conditions were: 98° C. for 10 sec, 60° C. for 45 sec, and 72° C. for 5 min 30 sec, for 15 cycles. The final 11 kb full length YF-DD was gel checked. Full length products were further purified by DNAland Gel Extraction PCR Purification 2-in-1 Kit.

### Diagnostic PCR Check

16 diagnostic PCRs were used to confirm that the F2-DD PCR building block as well as the final full length YF-DD DNA genome carry the intended deoptimized F2 sequence, and rule out presence of 17D sequence in the F2 region (E domain).

### RNA Synthesis

HiScribe™ T7 In Vitro Transcription Kit (NEB) were used to generate full length YF-DD RNA. 2 ul of GTP, UTP, CTP (each at 100 mM concentration, 0.4 ul of ATP (100 mM), 4 ul 40 mM m7G(5′)ppp(5′) RNA Cap Structure Analog (NEB) wer NA synthesis set at 37° C. for 3 hours. 2 ul of RNA were gel checked.

### Transfection

In vitro synthesized YF-DD RNA was used in transfection. Vero cells, seeded on 4×35 mm dishes. For transfection, 3 ul/7 ul RNA were mixed with 3.5 ul/7 ul Lipofectamine MessengerMAX mRNA Transfection Reagent for 5 min, and transferred to Vero cells grown in DMEM+ OptiPRO. Mock transfected dishes received the same amount of Lipofectamine, without RNA. Medium were changed every 2-3 days until Day 12 post transfection. Cell death were monitored daily.

### Virus Passage

Supernatants from Day 4, Day 7 and Day 12 post transfection dishes were collected and used to infect fresh Vero Cells.

### YF Staining

To visualize YF-DD virus-infected cells, mouse monoclonal anti-Flavivirus Group Antigen Antibody, clone D1-4G2-4-15 (ATCC® HB-112), in conjunction with HRP-labeled goat anti-mouse secondary antibody and VECTOR VIP chromog ll monolayers on Day 12 post transfection, or Day 8 post infection.

### Results & Discussion

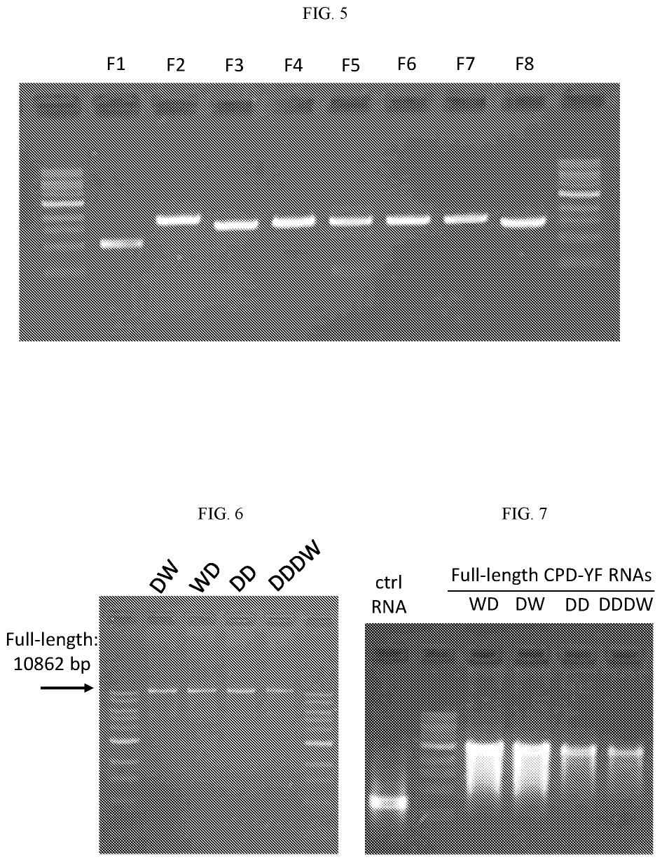

1. PCR for all 8 Fragment. All PCR reactions from original BioBasic plasmids were successful. All PCR products were purified by DNAland Gel Extraction PCR Purification 2-in-1 Kit. See FIG. 5.

2. Overlapping PCR for Full Length YF-DD. Full length YF-DD (11 kb) was successfully generated by overlapping PCR. Full length products were further purified by DNAland Gel Extraction PCR Purification 2-in-1 Kit. See FIG. 6

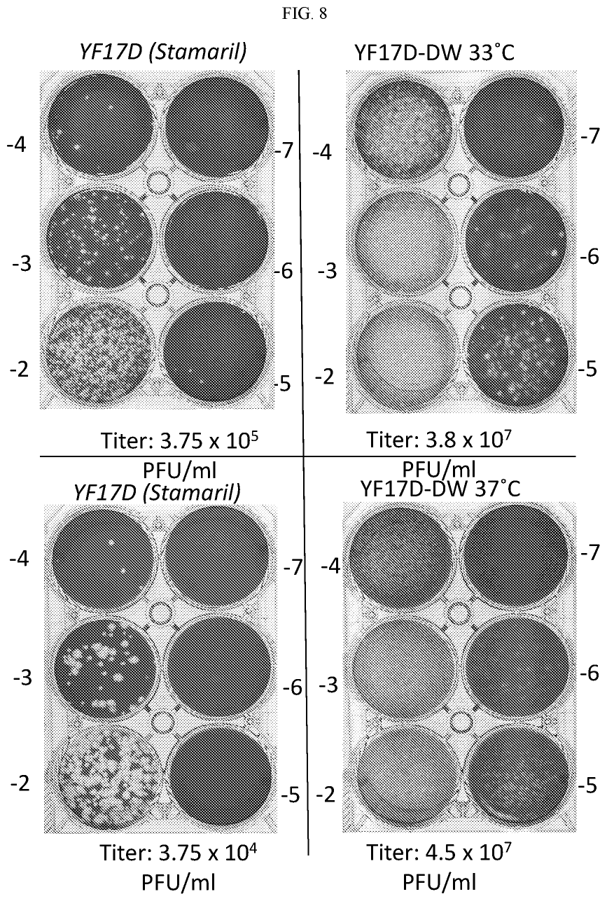

3. Diagnostic PCR Check. The first 8 diagnostic PCR check show correct pattern on both building block F2-DD (PCR product using in overlapping PCR) and full length YF-DD, indicating the first half of F2 region was correct deoptimized sequence without any WT contamination. FIG. 7. The second sets of 8 diagnostic PCR showed correct pattern on both building block F2-DD (PCR product using in overlapping PCR) and full length YF-DD, indicating the second half of F2 region was the correct deoptimized sequence without any WT contamination. FIG. 8.

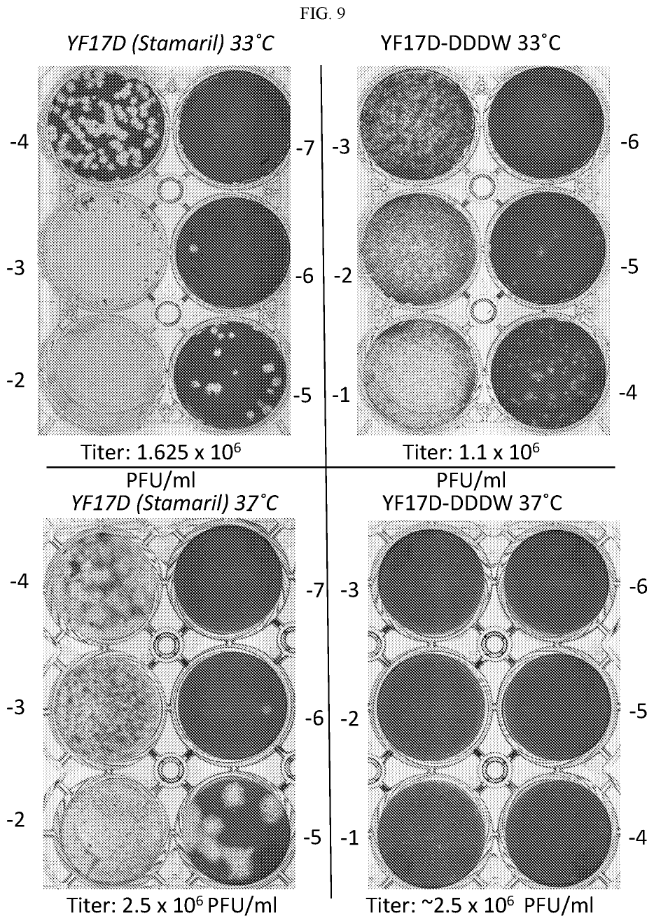

4. RNA synthesis. Full length Overlapping PCR YF-DD were used in RNA synthesis. RNA was evaluated before transfection. FIG. 9.

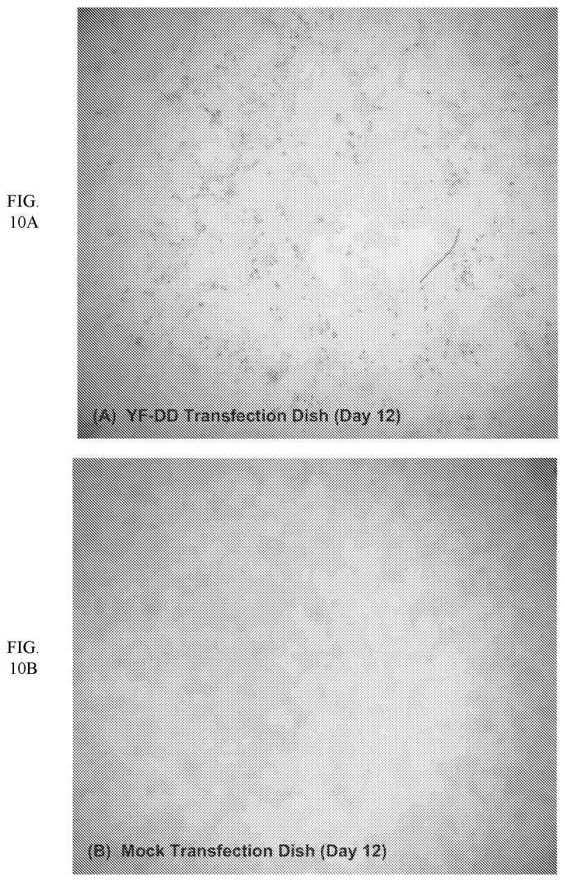

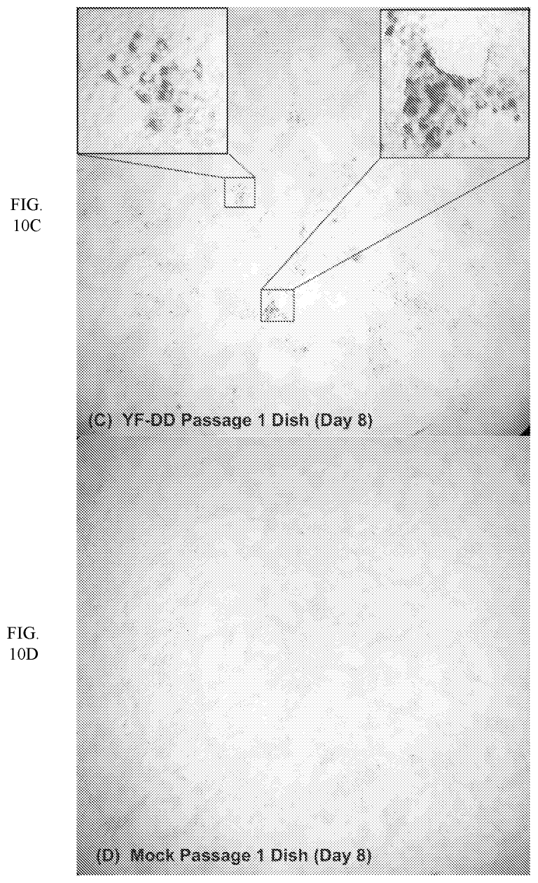

5. Detection of Yellow Fever Antigen by Immunohistochemical Staining of Transfected or Infected Cells. FIG. 10A-10D.

Yellow Fever Vaccine candidate YF-DD, which carries a wholly deoptimized E domain was successfully recovered by overlapping PCR and RNA transfection on Vero cells. Both the building block F2-DD and the full-length overlapping PCR products of YF-DD were PCR confirmed to carry the intended deoptimized DD sequence without detectable 17D sequence in the F2 region. Full length viral RNA was of high quality before transfection. The YF-DD virus was viable after transfection, as evidenced by a preponderance of infected cells upon immunohistochemical staining 12 days after RNA transfection.

YF-DD virus produced very little or no CPE after transfection. Blind passaging of the day 4 transfection harvest on fresh Vero cells confirmed the recovery of infectious YF-DD virus, as evidenced by a preponderance of newly infected cells upon immunohistochemical staining 8 days after infection (again without noticeable CPE). The absence of CPE is in stark contrast to the parental 17D virus under similar conditions (data not shown), indicating that YF-DD will likely be very highly attenuated.

The YF-DD virus is further passaged, titered and sequenced to prepare it for mouse neurovirulence testing.

Wild-type and Deoptimized Yellow Fever E Protein Coding Sequences

| | | SEQ ID |
|---|---|---|
| | Sequence | NO: |
| YF-Env- | ATGACTGGAAGAATGGGTGAAAGGCAACTCCAAAAGATTGAGA | 59 |
| Wt | GATGGTTCGTGAGGAACCCCTTTTTTGCAGTGACGGC | |
| | TCTGACCATTGCCTACCTTGTGGGAAGCAACATGACGCAACGAG | |
| | TCGTGATTGCCCTACTGGTCTTGGCTGTTGGTCCGG | |
| | CCTACTCAGCTCACTGCATTGGAATTACTGACAGGGATTTCATTG | |
| | AGGGGGTGCATGGAGGAACTTGGGTTTCAGCTACC | |
| | CTGGAGCAAGACAAGTGTGTCACTGTTATGGCCCCTGACAAGCC | |
| | TTCATTGGACATCTCACTAGAGACAGTAGCCATTGA | |
| | TAGACCTGCTGAGGTGAGGAAAGTGTGTTACAATGCAGTTCTCA | |
| | CTCATGTGAAGATTAATGACAAGTGCCCCAGCACTG | |
| | GAGAGGCCCACCTAGCTGAAGAGAACGAAGGGGACAATGCGTG | |
| | CAAGCGCACTTATTCTGATAGAGGCTGGGGCAATGGC | |
| | TGTGGCCTATTTGGGAAAGGGAGCATTGTGGCATGCGCCAAATT | |
| | CACTTGTGCCAAATCCATGAGTTTGTTTGAGGTTGA | |
| | TCAGACCAAAATTCAGTATGTCATCAGAGCACAATTGCATGTAG | |
| | GGGCCAAGCAGGAAAATTGGACTACCGACATTAAGA | |
| | CTCTCAAGTTTGATGCCCTGTCAGGCTCCCAGGAAGTCGAGTTCA | |
| | TTGGGTATGGAAAAGCTACACTGGAATGCCAGGTG | |
| | CAAACTGCGGTGGACTTTGGTAACAGTTACATCGCTGAGATGGA | |
| | AACAGAGAGCTGGATAGTGGACAGACAGTGGGCCCA | |
| | GGACTTGACCCTGCCATGGCAGAGTGGAAGTGGCGGGGTGTGGA | |
| | GAGAGATGCATCATCTTGTCGAATTTGAACCTCCGC | |
| | ATGCCGCCACTATCAGAGTACTGGCCCTGGGAAACCAGGAAGGC | |
| | TCCTTGAAAACAGCTCTTACTGGCGCAATGAGGGTT | |
| | ACAAAGGACACAAATGACAACAACCTTTACAAACTACATGGTGG | |
| | ACATGTTTCTTGCAGAGTGAAATTGTCAGCTTTGAC | |
| | ACTCAAGGGGACATCCTACAAAATATGCACTGACAAAATGTTTT | |
| | TTGTCAAGAACCCAACTGACACTGGCCATGGCACTG | |
| | TTGTGATGCAGGTGAAAGTGTCAAAAGGAGCCCCCTGCAGGATT | |
| | CCAGTGATAGTAGCTGATGATCTTACAGCGGCAATC | |
| | AATAAAGGCATTTTGGTTACAGTTAACCCCATCGCCTCAACCAA | |
| | TGATGATGAAGTGCTGATTGAGGTGAACCCACCTTT | |
| | TGGAGACAGCTACATTATCGTTGGGAGAGGAGATTCACGTCTCA | |
| | CTTACCAGTGGCACAAAGAGGGAAGCTCAATAGGAA | |
| | AGTTGTTCACTCAGACCATGAAAGGCGTGGAACGCCTGGCCGTC | |
| | ATGGGAGACACCGCCTGGGATTTCAGCTCCGCTGGA | |
| | GGGTTCTTCACTTCGGTTGGGAAAGGAATTCATACGGTGTTTGGC | |
| | TCTGCCTTTCAGGGGCTATTTGGCGGCTTGAACTG | |
| | GATAACAAAGGTCATCATGGGGGCGGTACTTATATGGGTTGGCA | |
| | TCAACACAAGAAACATGACAATGTCCATGAGCATGA | |
| | TCTTGGTAGGAGTGATCATGATGTTTTTGTCTCTAGGAGTTGGGG | |
| | CGGATCAAGGATGCGCCATCAACTTTGGCAAGAGA | |
| | GAGCTCAAGTGCGGAGATGGTATCTTCATATTTAGAGACTCTGA | |
| | TGACTGGCTGAACAAGTACTCATACTATCCAGAAGATCCTGTGA | |
| | AGCTTGCATCAATAGTGAAAGCCTCT | |
| YF-Env- | ATGACTGGAAGAATGGGTGAAAGGCAACTCCAAAAGATTGAGA | 60 |
| DW | GATGGTTCGTGAGGAACCCCTTTTTTGCAGTGACGGC | |
| | TCTGACCATTGCCTACCTTGTGGGAAGCAACATGACGCAACGAG | |
| | TCGTGATTGCCCTACTGGTCTTGGCTGTTGGTCCGG | |
| | CCTACTCAGCTCACTGCATTGGAATTACTGACAGGGATtttatcgaggg | |
| | ggtgcatggcggaacttgggttagcgctacactcgaacaggacaaatgcgttaccgttatggcccccgata | |
| | agcctagcctagacattagtctcgaaaccgttgcgatcgatagacccgccgaagtgagaaaagtgtgttata | |
| | acgccgtactgactcacgttaagattaacgacaaatgccctagtacaggcgaagcgcatctagccgaaga | |
| | gaacgagggcgataacgcatgcaaacgtacttatagcgatagggggtgggggaacggatgcggattgttc | |
| | ggtaaggggtcaatcgtcgcatgcgctaagtttacatgcgctaagtctatgtcattgttcgaagtcgatcaga | |
| | ctaagattcagtacgtgattagagcgcaattgcatgtgggagcgaaacaggagaattggactactgacatta | |
| | agacactgaaattcgacgcccttagcggatcacaggaggtcgagtttattgggtacggaaaagcgacactc | |
| | gagtgtcaggtgcagactgccgttgactttggcaattcatacatagccgaaatggagacagagtcatggatc | |
| | gttgacagacagtgggcccaggatctgacattgccatggcaatccggatccggaggcgtttggcgcgaaa | |
| | tgcatcatctagtcgagttcgaaccgccacatgccgctacaatcagagtgttggccctaggcaatcaggag | |
| | ggaTCCTTGAAAACAGCTCTTACTGGCGCAATGAGGGTT | |
| | ACAAAGGACACAAATGACAACAACCTTTACAAACTACATGGTGG | |
| | ACATGTTTCTTGCAGAGTGAAATTGTCAGCTTTGAC | |
| | ACTCAAGGGGACATCCTACAAAATATGCACTGACAAAATGTTTT | |
| | TTGTCAAGAACCCAACTGACACTGGCCATGGCACTG | |
| | TTGTGATGCAGGTGAAAGTGTCAAAAGGAGCCCCCTGCAGGATT | |
| | CCAGTGATAGTAGCTGATGATCTTACAGCGGCAATC | |
| | AATAAAGGCATTTTGGTTACAGTTAACCCCATCGCCTCAACCAA | |
| | TGATGATGAAGTGCTGATTGAGGTGAACCCACCTTT | |
| | TGGAGACAGCTACATTATCGTTGGGAGAGGAGATTCACGTCTCA | |
| | CTTACCAGTGGCACAAAGAGGGAAGCTCAATAGGAA | |
| | AGTTGTTCACTCAGACCATGAAAGGCGTGGAACGCCTGGCCGTC | |
| | ATGGGAGACACCGCCTGGGATTTCAGCTCCGCTGGA | |
| | GGGTTCTTCACTTCGGTTGGGAAAGGAATTCATACGGTGTTTGGC | |
| | TCTGCCTTTCAGGGGCTATTTGGCGGCTTGAACTG | |
| | GATAACAAAGGTCATCATGGGGGCGGTACTTATATGGGTTGGCA | |
| | TCAACACAAGAAACATGACAATGTCCATGAGCATGA | |
| | TCTTGGTAGGAGTGATCATGATGTTTTTGTCTCTAGGAGTTGGGG | |
| | CGGATCAAGGATGCGCCATCAACTTTGGCAAGAGA | |
| | GAGCTCAAGTGCGGAGATGGTATCTTCATATTTAGAGACTCTGA | |
| | TGACTGGCTGAACAAGTACTCATACTATCCAGAAGA | |
| | TCCTGTGAAGCTTGCATCAATAGTGAAAGCCTCT | |
| YF-Env- | ATGACTGGAAGAATGGGTGAAAGGCAACTCCAAAAGATTGAGA | 61 |
| WD | GATGGTTCGTGAGGAACCCCTTTTTTGCAGTGACGGC | |
| | TCTGACCATTGCCTACCTTGTGGGAAGCAACATGACGCAACGAG | |
| | TCGTGATTGCCCTACTGGTCTTGGCTGTTGGTCCGG | |
| | CCTACTCAGCTCACTGCATTGGAATTACTGACAGGGATTTCATTG | |
| | AGGGGGTGCATGGAGGAACTTGGGTTTCAGCTACC | |
| | CTGGAGCAAGACAAGTGTGTCACTGTTATGGCCCCTGACAAGCC | |
| | TTCATTGGACATCTCACTAGAGACAGTAGCCATTGA | |
| | TAGACCTGCTGAGGTGAGGAAAGTGTGTTACAATGCAGTTCTCA | |
| | CTCATGTGAAGATTAATGACAAGTGCCCCAGCACTG | |
| | GAGAGGCCCACCTAGCTGAAGAGAACGAAGGGGACAATGCGTG | |
| | CAAGCGCACTTATTCTGATAGAGGCTGGGGCAATGGC | |
| | TGTGGCCTATTTGGGAAAGGGAGCATTGTGGCATGCGCCAAATT | |
| | CACTTGTGCCAAATCCATGAGTTTGTTTGAGGTTGA | |
| | TCAGACCAAAATTCAGTATGTCATCAGAGCACAATTGCATGTAG | |
| | GGGCCAAGCAGGAAAATTGGACTACCGACATTAAGA | |
| | CTCTCAAGTTTGATGCCCTGTCAGGCTCCCAGGAAGTCGAGTTCA | |
| | TTGGGTATGGAAAAGCTACACTGGAATGCCAGGTG | |
| | CAAACTGCGGTGGACTTTGGTAACAGTTACATCGCTGAGATGGA | |
| | AACAGAGAGCTGGATAGTGGACAGACAGTGGGCCCA | |
| | GGACTTGACCCTGCCATGGCAGAGTGGAAGTGGCGGGGTGTGGA | |
| | GAGAGATGCATCATCTTGTCGAATTTGAACCTCCGC | |
| | ATGCCGCCACTATCAGAGTACTGGCCCTGGGAAACCAGGAAGGC | |
| | TCCcttaaaaccgcattgactggcgctatgcgcgttactaaggacactaacgacaataacctatacaaact | |
| | gcatggggggcatgtgtcttgtagagtgaaattgtccgcccttacacttaaggggactagctataagatatgc | |
| | actgacaaaatgtttttcgttaaaaaccctaccgataccggacacggaacagtcgttatgcaggtgaaagtgt | |
| | caaaaggcgcaccatgtaggatacccgtaatcgttgccgacgatctgactgccgcaatcaataaggggata | |
| | ctcgtgacagtgaaccctatcgctagcactaacgacgacgaagtgttgatcgaagtgaatccaccttttggc | |
| | gactcatacattatcgtaggcagaggcgatagtagactgacataccaatggcataaagagggatcgtcaat | |
| | cggtaagttgtttacacagactatgaaaggggtggagagattggccgttatgggcgataccgcttgggactt | |
| | tagttccgccggagggttttttactagcgtcggaaaggggatacataccgtattcggatccgcttttcagggg | |
| | ttgttcggcggactgaattggattacgaaagtgattatgggcgccgtacttatttgggtggggattaacactag | |
| | gaatatgactatgtctatgtctatgatactagtcggagtgattatgatgtttctgtcattgggcgtaggcgctG | |
| | ATCAAGGATGCGCCATCAACTTTGGCAAGAGA | |
| | GAGCTCAAGTGCGGAGATGGTATCTTCATATTTAGAGACTCTGA | |
| | TGACTGGCTGAACAAGTACTCATACTATCCAGAAGA | |
| | TCCTGTGAAGCTTGCATCAATAGTGAAAGCCTCT | |
| YF-Env- | ATGACTGGAAGAATGGGTGAAAGGCAACTCCAAAAGATTGAGA | 62 |
| DD | GATGGTTCGTGAGGAACCCCTTTTTTGCAGTGACGGC | |
| | TCTGACCATTGCCTACCTTGTGGGAAGCAACATGACGCAACGAG | |
| | TCGTGATTGCCCTACTGGTCTTGGCTGTTGGTCCGG | |
| | CCTACTCAGCTCACTGCATTGGAATTACTGACAGGGATtttatcgaggg | |
| | ggtgcatggcggaacttgggttagcgctacactcgaacaggacaaatgcgttaccgttatggcccccgata | |
| | agcctagcctagacattagtctcgaaaccgttgcgatcgatagacccgccgaagtgagaaaagtgtgttata | |
| | acgccgtactgactcacgttaagattaacgacaaatgccctagtacaggcgaagcgcatctagccgaaga | |
| | gaacgagggogataacgcatgcaaacgtacttatagcgatagggggtgggggaacgga | |
| | tgcggattgttcggtaaggggtcaatcgtcgcatgcgctaagtttacatgcgctaagtctatgtcattgttcga | |
| | agtcgatcagactaagattcagtacgtgattagagcgcaattgcatgtgggagcgaaacaggagaattgga | |
| | ctactgacattaagacactgaaattcgacgcccttagcggatcacaggaggtcgagtttattgggtacggaa | |
| | aagcgacactcgagtgtcaggtgcagactgccgttgactttggcaattcatacatagccgaaatggagaca | |
| | gagtcatggatcgttgacagacagtgggcccaggatctgacattgccatggcaatccggatccggaggcg | |
| | tttggcgcgaaatgcatcatctagtcgagttcgaaccgccacatgccgctacaatcagagtgttggccctag | |
| | gcaatcaggagggatcccttaaaaccgcattgactggcgctatgcgcgttactaaggacactaacgacaat | |
| | aacctatacaaactgcatggggggcatgtgtcttgtagagtgaaattgtccgcccttacacttaaggggacta | |
| | gctataagatatgcactgacaaaatgtttttcgttaaaaaccctaccgataccggacacggaacagtcgttat | |
| | gcaggtgaaagtgtcaaaaggcgcaccatgtaggatacccgtaatcgttgccgacgatctgactgccgca | |
| | atcaataaggggatactcgtgacagtgaaccctatcgctagcactaacgacgacgaagtgttgatcgaagt | |
| | gaatccaccttttggcgactcatacattatcgtaggcagaggcgatagtagactgacataccaatggcataa | |
| | agagggatcgtcaatcggtaagttgtttacacagactatgaaaggggtggagagattggccgttatgggcg | |
| | ataccgcttgggactttagttccgccggagggttttttactagcgtcggaaaggggatacataccgtattcgg | |
| | atccgcttttcaggggttgttcggcggactgaattggattacgaaagtgattatgggcgccgtacttatttggg | |
| | tggggattaacactaggaatatgactatgtctatgtctatgatactagtcggagtgattatgatgtttctgtcattg | |
| | ggcgtaggcgctGATCAAGGATGCGCCATCAACTTTGGCAAGAGA | |
| | GAGCTCAAGTGCGGAGATGGTATCTTCATATTTAGAGACTCTGA | |
| | TGACTGGCTGAACAAGTACTCATACTATCCAGAAGA | |
| | TCCTGTGAAGCTTGCATCAATAGTGAAAGCCTCT | |
| YF-Env- | ATGACTGGAAGAATGGGTGAAAGGCAACTCCAAAAGATTGAGA | 63 |
| DDDW | GATGGTTCGTGAGGAACCCCTTTTTTGCAGTGACGGC | |
| | TCTGACCATTGCCTACCTTGTGGGAAGCAACATGACGCAACGAG | |
| | TCGTGATTGCCCTACTGGTCTTGGCTGTTGGTCCGG | |
| | CCTACTCAGCTCACTGCATTGGAATTACTGACAGGGATtttatcgaggg | |
| | ggtgcatggcggaacttgggttagcgctacactcgaacaggacaaatgcgttaccgttatggcccccgata | |
| | agcctagcctagacattagtctcgaaaccgttgcgatcgatagacccgccgaagtgagaaaagtgtgttata | |
| | acgccgtactgactcacgttaagattaacgacaaatgccctagtacaggcgaagcgcatctagccgaaga | |
| | gaacgagggcgataacgcatgcaaacgtacttatagcgatagggggtgggggaacggatgcggattgttc | |
| | ggtaaggggtcaatcgtcgcatgcgctaagtttacatgcgctaagtctatgtcattgttcgaagtcgatcaga | |
| | ctaagattcagtacgtgattagagcgcaattgcatgtgggagcgaaacaggagaattggactactgacatta | |
| | agacactgaaattcgacgcccttagcggatcacaggaggtcgagtttattgggtacggaaaagcgacactc | |
| | gagtgtcaggtgcagactgccgttgactttggcaattcatacatagccgaaatggagacagagtcatggatc | |
| | gttgacagacagtgggcccaggatctgacattgccatggcaatccggatccggaggcgtttggcgcgaaa | |
| | tgcatcatctagtcgagttcgaaccgccacatgccgctacaatcagagtgttggccctaggcaatcaggag | |
| | ggatcccttaaaaccgcattgactggcgctatgcgcgttactaaggacactaacgacaataacctatacaaa | |
| | ctgcatggggggcatgtgtcttgtagagtgaaattgtccgcccttacacttaaggggactagctataagatat | |
| | gcactgacaaaatgtttttcgttaaaaaccctaccgataccggacacggaacagtcgttatgcaggtgaaag | |
| | tgtcaaaaggcgcaccatgtaggatacccgtaatcgttgccgacgatctgactgccgcaatcaataagggg | |
| | atactcgtgacagtgaaccctatcgctagcactaacgacgacgaagtgttgatcgaagtgaatccaccttt | |
| | tggcgactcatacattatcgtaggcagaggcgatagtagaCTCACTTACCAGTGGCACA | |
| | AAGAGGGAAGCTCAATAGGAA | |
| | AGTTGTTCACTCAGACCATGAAAGGCGTGGAACGCCTGGCCGTC | |
| | ATGGGAGACACCGCCTGGGATTTCAGCTCCGCTGGA | |
| | GGGTTCTTCACTTCGGTTGGGAAAGGAATTCATACGGTGTTTGGC | |
| | TCTGCCTTTCAGGGGCTATTTGGCGGCTTGAACTG | |
| | GATAACAAAGGTCATCATGGGGGCGGTACTTATATGGGTTGGCA | |
| | TCAACACAAGAAACATGACAATGTCCATGAGCATGA | |
| | TCTTGGTAGGAGTGATCATGATGTTTTTGTCTCTAGGAGTTGGGG | |
| | CGGATCAAGGATGCGCCATCAACTTTGGCAAGAGA | |
| | GAGCTCAAGTGCGGAGATGGTATCTTCATATTTAGAGACTCTGA | |
| | TGACTGGCTGAACAAGTACTCATACTATCCAGAAGA | |
| | TCCTGTGAAGCTTGCATCAATAGTGAAAGCCTCT | |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of generating a modified viral genome, comprising
performing reverse transcription polymerase chain reaction (“RT-PCR”) on a viral RNA from an RNA virus to generate cDNA;
performing polymerase chain reaction (“PCR”) to generate and amplify two or more overlapping cDNA fragments from the cDNA, wherein the two or more overlapping cDNA fragments collectively encode the RNA virus;
substituting one or more overlapping cDNA fragments comprising a modified sequence for one or more corresponding overlapping cDNA fragment generated from the viral RNA;
performing overlapping and amplifying PCR to construct the modified viral genome, wherein the modified viral genome comprises one or more modified sequences.

2. A method of generating a modified viral genome, comprising
performing polymerase chain reaction (“PCR”) to generate and amplify two or more overlapping cDNA fragments from cDNA encoding viral RNA from an RNA virus, wherein the two or more overlapping cDNA fragments collectively encode the RNA virus, wherein one or more overlapping cDNA fragments comprises a modified sequence;
performing overlapping and amplifying PCR to construct the modified viral genome, wherein the modified viral genome comprises one or more modified sequences.

3. The method of claim 1, further comprising extracting the viral RNA from the RNA virus prior to performing RT-PCR.

4. The method of claim 1, wherein each of the one or more overlapping cDNA fragments comprising the modified sequence comprises (1) a recoded sequence having reduced codon pair bias compared to a corresponding sequence on the cDNA, (2) an increased number of CpG or UpA di-nucleotides compared to a corresponding sequence on the cDNA; or (3) at least 5 codons substituted with synonymous codons less frequently used.

5. The method of claim 1, wherein performing PCR to generate and amplify two or more overlapping cDNA fragments from the cDNA comprises using two or more primer pairs selected from Table 1.

6. The method of claim 1, wherein the two or more overlapping cDNA fragments from the cDNA is 10 or more overlapping cDNA fragments and the 10 or more overlapping cDNA fragments collectively encode the RNA virus.

7. The method of claim 6, wherein performing PCR to generate and amplify 10 or more overlapping cDNA fragments from the cDNA comprises using 10 or more primer pairs selected from Table 1.

8. The method of claim 1, wherein the two or more overlapping cDNA fragments from the cDNA is 15 or more overlapping cDNA fragments and the 15 or more overlapping cDNA fragments collectively encode the RNA virus.

9. The method of claim 8, wherein performing PCR to generate and amplify 15 or more overlapping cDNA fragments from the cDNA comprises using 15 or more primer pairs selected from Table 1.

10. The method of claim 1, wherein the two or more overlapping cDNA fragments from the cDNA is 19 overlapping cDNA fragments and the 19 overlapping cDNA fragments collectively encode the RNA virus.

11. The method of claim 10, wherein performing PCR to generate and amplify 19 overlapping cDNA fragments from the first cDNA comprises using all 19 primer pairs from Table 1.

12. The method of claim 1, wherein the viral RNA is from a wild-type RNA virus, and the cDNA is cDNA encoding the viral RNA from the wild-type RNA virus (“wild-type cDNA”).

13. The method of claim 1, wherein the viral RNA is from SARS-CoV-2, SARS-CoV-2 variant, or Yellow Fever virus.

14. The method of claim 1, wherein each of the primers are about 15-65 base pairs (bp) in length.

15. The method of claim 1, wherein each of the primers are about 15-55 base pairs (bp) in length.

16. The method of claim 1, wherein each overlap between the two or more overlapping cDNA fragments overlap by about 40-400 bp.

17. The method of claim 1, wherein each overlap between the two or more overlapping cDNA fragments overlap by about 100-300 bp.

18. The method of claim 1, comprising
performing RT-PCR on viral RNA from a wild-type RNA virus to generate cDNA (“wild-type cDNA”);
performing PCR to generate and amplify 19 overlapping cDNA fragments from the wild-type cDNA, wherein the 19 overlapping cDNA fragments collectively encode the wild-type RNA virus;
substituting an overlapping cDNA fragment comprising a deoptimized sequence for a corresponding overlapping cDNA fragment from the wild-type cDNA; and
performing overlapping and amplifying PCR to construct the modified viral genome comprising the deoptimized sequence.

19. A method of generating a modified infectious RNA, comprising:
performing in vitro transcription of a modified viral genome to generate a modified RNA transcript.

20. The method of claim 19, further comprising
performing reverse transcription polymerase chain reaction (“RT-PCR”) on a viral RNA from an RNA virus to generate cDNA;
performing polymerase chain reaction (“PCR”) to generate and amplify two or more overlapping cDNA fragments from the cDNA, wherein the two or more overlapping cDNA fragments collectively encode the RNA virus;
substituting one or more overlapping cDNA fragments comprising a modified sequence for one or more corresponding overlapping cDNA fragment generated from the viral RNA; and
performing overlapping and amplifying PCR to construct the modified viral genome, wherein the modified viral genome comprises one or more modified sequences, to generate the modified viral genome before performing the in vitro transcription.

21. A method of generating a modified virus, comprising
transfecting host cells with a quantity of a modified infectious RNA;
culturing the host cells; and
collecting infection medium comprising the modified virus.

22. The method of claim 21, further comprising
performing in vitro transcription of a modified viral genome to generate a modified RNA transcript to obtain the quantity of modified infectious RNA before transfecting host cells with the quantity of the modified infectious RNA.
